(19) United States
(12) Patent Application Publication
(10) Pub. No.: US 2017/0266273 A1
(43) Pub. Date: Sep. 21, 2017

(54) RECOMBINANT ADENO-ASSOCIATED VIRUS VECTORS CARRYING THE MUTANT HPV- 16 E7 ANTIGEN GENE, CONSTRUCTION METHOD AND APPLICATION THEREOF

(71) Applicant: GUANGDONG TOPHEALTH BIOTECHNOLOGY CO., LTD., Guangzhou, Guangdong (CN)

(72) Inventors: Yong LIU, Guangzhou, Guangdong (CN); Qiaolin CHEN, Guangzhou, Guangdong (CN); Zhaopeng ZENG, Guangzhou, Guangdong (CN); Wenjuan DONG, Guangzhou, Guangdong (CN); Hongji GAO, Guangzhou, Guangdong (CN); Yanhao GONG, Guangzhou, Guangdong (CN); Sha MENG, Guangzhou, Guangdong (CN); Yanwei XU, Guangzhou, Guangdong (CN); Hui ZHANG, Guangzhou, Guangdong (CN); Jing LU, Guangzhou, Guangdong (CN)

(21) Appl. No.: 15/501,047

(22) PCT Filed: Aug. 12, 2015
(86) PCT No.: PCT/CN2015/086809

§ 371 Date: Feb. 1, 2017

(30) Foreign Application Priority Data
Aug. 1, 2014 (CN) 201410377605.2
Aug. 12, 2014 (CN) 201410395785.7
Aug. 12, 2014 (CN) 201410395805.0
Aug. 12, 2014 (CN) 201410395934.X

(51) Int. Cl. A61K 39/12; A61K 48/00; A61K 39/00; C12N 15/86; C07K 14/005

(52) U.S. Cl. CPC A61K 39/12; C12N 15/86; C07K 14/005; A61K 39/0011; A61K 48/005; C12N 2750/14143; C12N 2710/20022; C12N 2710/20034; A61K 2039/585; A61K 2039/5154; A61K 2039/572; C12N 2800/24

(57) ABSTRACT

Provided are a recombinant adeno-associated virus (AAV) vector carrying human papillomavirus type 16 (HPV-16) mutation E7 antigen gene and a construction method therefor. The construction method comprises mutating the carcinogenic HPV-16 E7 antigen gene to noncarcinogenic E7 antigen gene, and then inserting the mutated gene into an AAV vector of which the structural gene has been removed, thereby obtaining the recombinant AAV vector. The recombinant AAV vector or related product can be used for preparing medications against HPV-16 infection and other diseases such as tumors caused by HPV-16 infection.

This application is the U.S. national phase of International Application No. PCT/CN2015/086809 filed 12 Aug. 2015, which designated the U.S. and claims priority to CN Patent Application Nos. 201410377605.2 filed 1 Aug. 2014, 201410395785.7 filed 12 Aug. 2014, 201410395805.0 filed 12 Aug. 2014, and 201410395934.X filed 12 Aug. 2014, the entire contents of each of which are hereby incorporated by reference.

### TECHNICAL FIELD

The present invention involves in vectors and application thereof in biological field, especially involves in a recombinant adeno-associated virus vector (rAAV) carrying the single- or multiple-site mutant E7 antigen gene of human papillomavirus type 16 (HPV-16), construction method thereof and application thereof in producing medicine for anti-HPV-16 infection and HPV-16-infection associated diseases (for example, tumor caused by HPV-16 infection).

### BACKGROUND ART

The gene structure of adeno-associated virus (AAV) had been identified. Samulski, et al. described the terminal repeat fragment of AAV: upstream 5′-fragment, downstream 3′-fragment in 1983 (Samulski R J, Srivastava A, Berns K I, Muzyczka N. Rescue of adeno-associated virus from recombinant plasmids: gene correction within the terminal repeats of AAV. Cell. 33:135-143.). Hermonat, et al. described the low infectious particle (Lip) gene and capsule (Cap) gene of AAV in 1984 (Hermonat P L, Labow M A, Wright R, Berns K I, Muzyczka N. Genetics of adeno-associated virus: isolation and preliminary characterization of adeno-associated virus type 2 mutants. J Virol. 51:329-339. Hermonat, P. L., and Muzyczka, N. Use of adeno-associated virus as a mammalian DNA cloning vector: transduction of neomycin resistance into mammalian tissue culture cells. Proc. Natl. Acad. Sci. U.S.A. 81: 6466-6470.). Labow, et al. identified the p5 promoter between the upstream 5′-fragment and the replication protein (Rep) gene in 1986 (Labow M A, Hermonat P L, Berns K I. Positive and negative autoregulation of the adeno-associated virus type 2 genome. J Virol. 160:251-258.).

AAV is a nonpathogenic defective virus which needs the assistance of gene product of other virus (such as adeno virus) to assemble into the infectious virus particles. The full length of the AAV genome is about 4700 base pair (bp). Both ends of the AAV genome are terminal repeat fragment (TR), and the structural genes including replication-associated Rep gene and Cap gene are in the middle of the genome. It is necessary to construct recombinant adeno-associated virus (rAAV) by gene recombination due to the instability of AAV itself and the limitation of length of the exogenous genes (therapeutic gene). At present, a large number of researches reveal that the exogenous gene capacity of AAV can be significantly increased by deleting some structural genes in its genome. Moreover, infectious rAAV particles can be prepared by inserting the therapeutically exogenous genes into rAAV.

In 1984, Paul L. Hermonat originally proved that AAV vector could be used to the gene therapy of human diseases (Hermonat, P. L., and Muzyczka, N. Use of adeno-associated virus as a mammalian DNA cloning vector: transduction of neomycin resistance into mammalian tissue culture cells. Proc. Natl. Acad. Sci. U.S.A. 81: 6466-6470.). At present, the clinical trials of the gene therapy of human diseases based on AAV are mainly being conducted in Europe and America. According to the statistics of U. S. Food and Drug Administration, more than 10 clinical trials of the gene therapy of human diseases based on AAV are being conducted. These clinical trials are mainly about treating diseases (non-neoplastic diseases such as Parkinsonism disease, rheumarthritis, hemophilia, cardiac failure, cruveilhier's atrophy, Alzheimer's disease and so on) by injecting AAV with therapeutic gene into the patients and making the therapeutic gene to be expressed in vivo. Glybera produced by UniQure Company, which is the firstly approved gene therapy drug in western countries, was approved in 27 member states by European Union on Nov. 2, 2012. Glybera is used in the treatment of lipoprotein lipase deficiency (LPLD) by the adeno-associated virus type 1 carrying exogenous genes.

Human papillomavirus (HPV) belongs to Papovavirus genus A of Papovaviridae. There are more than 130 subtypes of HPV, and they are divided into two types: high-risk HPV and low-risk HPV. The most harmful HPV to humans is the high-risk HPV, including HPV-16, HPV-18, HPV-30, HPV-31, HPV-33, HPV-35 and HPV-39 which are closely associated with malignant tumors such as cervical cancer, rectal cancer, oral cancer, tonsillar cancer and so on. Over 99% of the cervical cancers are caused by high-risk HPV, and over half of the cervical cancers are caused by HPV-16.

HPV is a Parvoviridae with double-stranded closed circular DNA, including about 8000 basic pairs. There are 8 early open reading frames (E1-E8), 2 late open reading frames and one non-coding region. Among the early open reading frames, the cancerogenic E6 gene and E7 gene are the most important to cell growth stimulation. The E6 and E7 oncoprotein encoded by E6 and E7 gene respectively combine to cancer suppressor gene p53 and Rb respectively, which leads to the deregulation of cell proliferation, the disfunction of cancer suppressor genes for DNA damage repair, the precancerous lesion and the consequent cancers.

An investigation result (2003-2004) from American national research subject of health and nutrition showed that the total infection rate of HPV was 26.8% among women aged 14 to 59. Prevalence of HPV infection in China has not been officially reported. There are more than 200,000 newly added cervical cancer cases every year in China. There is an increasing trend of morbidity and mortality of cervical cancer. The incidence age of patients with cervical cancer is becoming younger. A less optimistic prospect of HPV infection can be inferred. However, exact treatment method for HPV infection has not been found yet. Although the present HPV vaccines are likely to prevent HPV-16 and HPV-18 infection, they are ineffective for infected people. For curing HPV infection, the optimal treatment is complete elimination of all the infected cells. The HPV-16 E7 antigen is an excellent target for cellular immunotherapy, because HPV-16 E7 antigen exists in all infected cells. However, HPV-16 E7 antigen is a carcinogenic protein which plays a leading role in the occurrence of malignant tumor, such as cervical cancer. Certain risk in terms of security exists when using wild-type HPV-16 E7 antigen to stimulate the immunoreaction in vitro and in vivo. Therefore, to eliminate the risk, the oncogenicity of wild-type HPV-16 E7 antigen must be removed.

Human dendritic cells (DC) are the main and most important antigen presenting cells. A large number of researches indicate that no matter in vitro or in vivo, DC can induce or stimulate the occurrence of anti-infectious and antineoplastic immunoreaction.

### DISCLOSURE OF THE INVENTION

One object of the present invention is to provide recombinant adeno-associated virus (rAAV) vectors carrying the nonpathogenic (i.e., non-oncogenic) mutant Human papillomavirus type 16 (HPV-16) E7 antigen gene.

The recombinant adeno-associated virus vector provided in the present invention is new rAAV vector obtained by inserting a mutant HPV-16 E7 antigen gene into an original vector; wherein the original vector is an AAV vector from which the adeno-associated virus structural genes Rep and Cap in the adeno-associated virus vector have been eliminated and by which any one of AAV p5 promoter, cytomegalovirus (CMV) promoter, SV40 virus promoter and β-actin promoter (β-actin) is carried. The recombinant adeno-associated virus vector is a recombinant adeno-associated virus vector carrying mutant HPV-16 E7 antigen gene.

Herein, the mutant HPV-16 E7 antigen genes are obtained by mutating the HPV-16 E7 antigen gene by molecular biotechnology, i. e., one, two or three of the cysteine (C) at position 58th, 91th and 94th of the E7 antigen protein of HPV-16 (American NCI gene pool No.: KC935953) are replaced with glycine (G) by replacing one, two or three of thymine (T) at position nt175, nt271 and nt280 (locations in Sequence Listing, respectively correspond to nt736, nt832 and nt841 in FIG. 3A-3G) of the open reading frames of HPV-16 E7 antigen gene with guanine (G), to obtain mutant HPV-16 E7 antigen gene (named as “HPV-16 E7ₘ”) that can express non-oncogenicity.

The mutant HPV-16 E7 antigen gene can be inserted into an adeno-associated virus vector (AAV), which carries any one of AAV p5 promoter, cytomegalovirus promoter, SV40 virus promoter and β-actin promoter, to obtain a recombinant adeno-associated virus vector (named as AAV/HPV-16 E7ₘ) that carrying the mutant HPV-16 E7 antigen gene, since the immunogenicity of the mutant HPV-16 E7 antigen gene is not influenced.

In particular, the mutant HPV-16 E7 antigen gene is one of the following genes (in combination with the Sequence Listing):

HPV-16 E7ₘ₅₈ gene: a mutant HPV-16 E7 gene carrying one mutation site of nt175 (58aa), named as HPV-16 E7ₘ₅₈. FIG. 3A (italic in nt736 is the mutation) shows the sequence alignment of HPV-16 E7ₘ₅₈ gene with HPV-16 E7 antigen gene. SEQ ID NO: 1 in Sequence Listing shows the nucleotide sequence of HPV-16 E7 antigen gene, and SEQ ID No: 2 in Sequence Listing shows the nucleotide sequence of HPV-16 E7ₘ₅₈ gene.

HPV-16 E7ₘ₉₁ gene: a mutant HPV-16 E7 gene carrying one mutation site of nt271 (91aa) named as HPV-16 E7ₘ₉₁. FIG. 3B (italic in nt832 is the mutation) shows the sequences alignment of HPV-16 E7ₘ₉₁ gene with HPV-16 E7 antigen gene. SEQ ID NO: 3 in Sequence Listing shows the nucleotide sequence of HPV-16 E7ₘ₉₁ gene.

HPV-16 E7ₘ₉₄ gene: a mutant HPV-16 E7 gene carrying one mutation site of nt280 (94aa) named as HPV-16 E7ₘ₉₄. FIG. 3C (italic in nt841 is the mutation) shows the sequences alignment of HPV-16 E7ₘ₉₄ gene with HPV-16 E7 antigen gene. SEQ ID NO: 4 in Sequence Listing shows the nucleotide sequence of HPV-16 E7ₘ₉₄ gene.

HPV-16 E7ₘₘ₂₁ gene: a multiple-site mutant HPV-16 E7 gene carrying two mutation sites of nt175 (58aa) and nt271 (91aa), and named as HPV-16 E7ₘₘ₂₁. FIG. 3D (italic in nt736 and nt832 are the mutations) shows the sequences alignment of HPV-16 E7ₘₘ₂₁ gene with HPV-16 E7 antigen gene. SEQ ID NO: 5 in Sequence Listing shows the nucleotide sequence of HPV-16 E7ₘₘ₂₁ gene.

HPV-16 E7ₘₘ₂₂ gene: a multiple-site mutant HPV-16 E7 gene carrying two mutation sites of nt175 (58aa) and nt280 (94aa), and named as HPV-16 E7ₘₘ₂₂. FIG. 3E (italic in nt736 and nt841 are the mutations) shows the sequences alignment of HPV-16 E7ₘₘ₂₂ gene with HPV-16 E7 antigen gene. SEQ ID NO: 6 in Sequence Listing shows the nucleotide sequence of HPV-16 E7ₘₘ₂₂ gene.

HPV-16 E7ₘₘ₂₃ gene: a multiple-site mutant HPV-16 E7 gene carrying two mutation sites of nt271 (91aa) and nt280 (94aa), and named as HPV-16 E7ₘₘ₂₃. FIG. 3F (italic in nt832 and nt841 are the mutations) shows the sequences alignment of HPV-16 E7ₘₘ₂₃ gene with HPV-16 E7 antigen gene. SEQ ID NO: 7 in Sequence Listing shows the nucleotide sequence of HPV-16 E7ₘₘ₂₃ gene.

HPV-16 E7ₘₘ₃ gene: a multiple-site mutant HPV-16 E7 gene carrying three mutation sites of nt175 (58aa), nt271 (91aa) and nt280 (94aa), and named as HPV-16 E7ₘₘ₃. FIG. 3G (italic in nt736, nt832 and nt841 are the mutations) shows the sequences alignment of HPV-16 E7ₘₘ₃ gene with HPV-16 E7 antigen gene. SEQ ID NO: 8 in Sequence Listing shows the nucleotide sequence of HPV-16 E7ₘₘ₃ gene.

The above-mentioned design also can be used to insert wild-type HPV-16 E7 antigen gene into adeno-associated virus vector to obtain recombinant adeno-associated virus vectors (named as AAV/HPV-16 E7) carrying wild-type HPV-16 E7 antigen gene. SEQ ID NO: 1 in Sequence Listing shows the nucleotide sequence of the wild-type HPV-16 E7 antigen gene. The present invention is recommended to research instead of clinical practice because of the oncogenicity of the wild-type HPV-16 E7 antigen.

Another object of the present invention is to provide a construction method of the above-mentioned recombinant adeno-associated virus vectors, AAV/HPV-16 E7 and AAV/HPV-16 E7ₘ. The construction method includes elimination of adeno-associated virus structural genes Rep and Cap in the adeno-associated virus vector and subsequent replacement of the eliminated genes with the above-mentioned HPV-16 E7 gene or the mutant HPV-16 E7ₘ gene by using the conventional gene recombination method to obtain the recombinant adeno-associated virus vector, AAV/HPV-16 E7 or AAV/HPV-16 E7ₘ.

The construction method provided by the present invention comprises the following steps:

1) By use of a conventional method in molecular biotechnology, HPV-16 E7 antigen gene is obtained and then mutated, i.e., one, two or three of cysteine (C) at position 58, 91 and 94 of the HPV-16 E7 antigen is (are) replaced with glycine (G). The detailed procedure is to replace one, two or three of thymine (T) at position nt175, nt271 and nt280 of the open reading frames of HPV-16 E7 gene with guanine (G) to obtain a mutant HPV-16 E7 antigen gene with one, two or three mutation sites (uniformly named as HPV-16 E7ₘ);

2) The mutant HPV-16 E7ₘ antigen gene or the wild-type HPV-16 E7 antigen gene is respectively inserted into an adeno-associated virus vector from which the AAV structural genes Rep and Cap have been eliminated to obtain a recombinant adeno-associated virus vector carrying the mutant HPV-16 E7ₘ antigen gene (AAV/HPV-16 E7ₘ) or a recombinant adeno-associated virus vector carrying the HPV-16 E7 antigen gene (AAV/HPV-16 E7).

The transcription promoter for HPV-16 E7ₘ or HPV-16 E7 antigen gene in the above-mentioned vectors can be any one of AAV p5 promoter, cytomegalovirus (CMV) promoter, β-actin promoter or SV40 virus early promoter or β-actin promoter.

The corresponding recombinant adeno-associated virus vector carrying mutant HPV-16 E7ₘ antigen gene, AAV/HPV-16 E7ₘ, include the following seven forms:

1. Cysteine (C) at position 58 of the wild-type HPV-16 E7 antigen protein is replaced with glycine (G). The detailed procedure is to replace the thymine (T) at position nt175 (location in Sequence Listing, corresponds to nt736 in FIG. 3A) of the open reading frames of HPV-16 E7 antigen gene with guanine (G), i. e., to replace the “tgc” encoding cysteine (nt175-177) with “ggc” encoding glycine, to obtain the mutant HPV-16 E7 antigen gene which can express non-oncogenicity and is named as HPV-16 E7ₘ₅₈ gene. Then the mutant HPV-16 E7ₘ₅₈ antigen gene is inserted into an adeno-associated virus from which the adeno-associated virus structural genes Rep and Cap have been eliminated to obtain a recombinant adeno-associated virus vector which carries the mutant HPV-16 E7ₘ₅₈ antigen gene and is named as AAV/HPV-16 E7ₘ₅₈.

2. Cysteine (C) at position 91 of the wild-type HPV-16 E7 antigen protein is replaced with glycine (G). The detailed procedure is to replace the thymine (T) at position nt271 (location in Sequence Listing, corresponds to nt832 in FIG. 3B) of the open reading frames of HPV-16 E7 antigen gene with guanine (G), i. e., to replace the “tgc” encoding cysteine (nt271-273) with “ggc” encoding glycine, to obtain the mutant HPV-16 E7 antigen gene which can express non-oncogenicity and is named as HPV-16 E7ₘ₉₁gene. Then the mutant HPV-16 E7ₘ₉₁ antigen gene is inserted into an adeno-associated virus from which the adeno-associated virus structural genes Rep and Cap have been eliminated to obtain a recombinant adeno-associated virus vector which carries the mutant HPV-16 E7ₘ₉₁ antigen gene and is named as AAV/HPV-16 E7ₘ₉₁.

3. Cysteine (C) at position 94 of the wild-type HPV-16 E7 antigen protein is replaced with glycine (G). The detailed procedure is to replace the thymine (T) at position nt280 (location in Sequence Listing, corresponds to nt841 in FIG. 3C) of the open reading frames of HPV-16 E7 antigen gene with guanine (G), i. e., to replace the “tgt” encoding cysteine (nt280-282) with “ggt” encoding glycine, to obtain the mutant HPV-16 E7 antigen gene which can express non-oncogenicity and is named as HPV-16 E7ₘ₉₄. Then the mutant HPV-16 E7ₘ₉₄ antigen gene is inserted into an adeno-associated virus from which the adeno-associated virus structural genes Rep and Cap have been eliminated to obtain a recombinant adeno-associated virus vector which carries the mutant HPV-16 E7ₘ₉₄ antigen gene and is named as AAV/HPV-16 E7ₘ₉₄.

4. Cysteines (C) at position 58 and 91 of the wild-type HPV-16 E7 antigen protein is replaced with glycines (G). The detailed procedure is to replace the thymine (T) at position nt175 and nt271 of the open reading frames of HPV-16 E7 antigen gene with guanine (G), i. e., to replace the “tgc” encoding cysteine (nt175-177 and nt271-273) with “ggc” encoding glycine, to obtain the mutant HPV-16 E7 antigen gene which has two mutation sites and is named as HPV-16 E7ₘₘ₂₁. Then the multiple-site-mutant HPV-16 E7ₘₘ₂₁ antigen gene is inserted into an adeno-associated virus from which the adeno-associated virus structural genes Rep and Cap have been eliminated to obtain a recombinant adeno-associated virus vector which carries the multiple-site-mutant HPV-16 E7ₘₘ₂₁ antigen gene and is named as AAV/HPV-16 E7ₘₘ₂₁.

5. Cysteines (C) at position 58 and 94 of the wild-type HPV-16 E7 antigen protein is replaced with glycines (G). The detailed procedure is to replace the two thymines (T) at position nt175 and nt280 of the open reading frames of HPV-16 E7 antigen gene with guanine (G), i. e., to replace the “tgc” (nt175-177) encoding cysteine at position 58 with “ggc” encoding glycine and to replace the “tgt” (nt280-282) encoding cysteine at position 94 with “ggt” encoding glycine, to obtain the mutant HPV-16 E7 antigen gene which has two mutation sites and is named as HPV-16 E7ₘₘ₂₂. Then the multiple-site-mutant HPV-16 E7ₘₘ₂₂ antigen gene is inserted into an adeno-associated virus from which the adeno-associated virus structural genes Rep and Cap have been eliminated to obtain a recombinant adeno-associated virus vector which carries the multiple-site-mutant HPV-16 E7ₘₘ₂₂ antigen gene and is named as AAV/HPV-16 E7ₘₘ₂₂.

6. Cysteines (C) at position 91 and 94 of the wild-type HPV-16 E7 antigen protein is replaced with glycines (G). The detailed procedure is to replace the two thymines (T) at position nt271 and nt280 of the open reading frames of HPV-16 E7 antigen gene with guanine (G), i. e., to replace the “tgc” (nt271-273) encoding cysteine at position 91 with “ggc” encoding glycine and to replace the “tgt” (nt280-282) encoding cysteine at position 94 with “ggt” encoding glycine, to obtain the mutant HPV-16 E7 antigen gene which has two mutation sites and is named as HPV-16 E7ₘₘ₂₃. Then the multiple-site-mutant HPV-16 E7ₘₘ₂₃ antigen gene is inserted into an adeno-associated virus from which the adeno-associated virus structural genes Rep and Cap have been eliminated to obtain a recombinant adeno-associated virus vector which carries the multiple-site-mutant HPV-16 E7ₘₘ₂₃ antigen gene and is named as AAV/HPV-16 E7ₘₘ₂₃.

7. Cysteines (C) at position 58, position 91 and 94 of the wild-type HPV-16 E7 antigen protein are replaced with glycines (G). The detailed procedure is to replace the three thymines (T) at position nt175, nt271 and nt280 of the open reading frames of HPV-16 E7 antigen gene with guanine (G), i. e., to replace the “tgc” (nt175-177 and nt271-273) encoding cysteine at position 58 and 91 with “ggc” encoding glycine and to replace the “tgt” (nt280-282) encoding cysteine at position 94 with “ggt” encoding glycine, to obtain the mutant HPV-16 E7 antigen gene which has three mutation sites and is named as HPV-16 E7ₘₘ₃. Then the multiple-site-mutant HPV-16 E7ₘₘ₃ antigen gene is inserted into an adeno-associated virus from which the adeno-associated virus structural genes Rep and Cap have been eliminated to obtain a recombinant adeno-associated virus vector which carries the multiple-site-mutant HPV-16 E7ₘₘ₃ antigen gene and is named as AAV/HPV-16 E7ₘₘ₃.

Another object of the present invention is to provide products associated with the recombinant adeno-associated virus vectors, AAV/HPV-16 E7ₘ and AAV/HPV-16 E7. The products include recombinant adeno-associated virus plasmids, recombinant adeno-associated virus particles and cell lines infected or transfected by the recombinant adeno-associated virus vector. The cell lines include monocytes and dendritic cells (DC) lines. The associated gene, HPV-16 E7ₘ antigen gene or HPV-16 E7 antigen gene, in the adeno-associated virus vector can be expressed in monocytes or dendritic cells via the above-mentioned transcription promoters.

The preparation methods of the said products associated with the recombinant adeno-associated virus vectors, AAV/HPV-16 E7 and AAV/HPV-16 E7ₘ, are respectively as follows:

Preparation of the recombinant adeno-associated virus plasmids: DNA of the recombinant adeno-associated virus vector, AAV/HPV-16 E7 or AAV/HPV-16 E7ₘ, are respectively introduced into genetically engineered Escherichia coli DH5α competent cells. And resistance screening is performed by using LB agar plates with 100 μg/mL ampicillin and the white colonies are picked, then plasmids of the picked colonies are extracted and purified to obtain the AAV/HPV-16 E7 plasmids and AAV/HPV-16 E7ₘ plasmids.

Preparation of the recombinant adeno-associated virus: The pHelper plasmids and the recombinant adeno-associated virus plasmids, AAV/HPV-16 E7 plasmids or AAV/HPV-16 E7ₘ plasmids, are used to co-transfect AAV-HEK293 cells to obtain the AAV, named as AAV/HPV-16 E7 virus and AAV/HPV-16 E7ₘ virus respectively.

Preparation of the cell lines infected or transfected by the recombinant adeno-associated virus: The said recombinant adeno-associated virus, AAV/HPV-16 E7 virus or AAV/HPV-16 E7ₘ virus, are used to infect or transfect monocytes, dendritic cells or lymphocytes respectively or in turn to obtain the cell lines.

In terms of practical use, it is another object of the present invention to provide a cellular immunotherapy medicine for anti-HPV-16infection and a malignant tumor caused by HPV-16 infection and the associated technique thereof.

The active ingredient of the said medicine is the above-mentioned recombinant adeno-associated virus vectorcarrying the HPV-16 E7ₘ antigen gene, AAV/HPV-16 E7ₘ, or the products associated with the AAV/HPV-16 E7ₘ of the present invention (pharmaceutical use of the wild-type HPV-16 E7 antigen is not considered due to its oncogenicity).

With the recombinant adeno-associated virus of present invention as a vector, the mutant HPV-16 E7ₘ gene can be introduced into monocytes to induce the production of dendritic cells or can be directly introduced into dendritic cells to express HPV-16 E7ₘ antigen protein, to achieve the aim of stimulating the immune response of the patient in vivo or in vitro to treat HPV-16 infection and associated malignant tumor caused by HPV-16 infection; or cytotoxic-T-lymphocytes (CTL) produced by stimulation of the dendritic cells may be used to treat HPV-16 infection and malignant tumor caused by HPV-16 infection.

The said malignant tumor caused by HPV-16 infection includes HPV-16-E7-antigen-positive cervical papilloma lesions, cervical cancer, male genital Bowen's disease, giant condyloma accuminata, penile cancer, anal cancer, rectum cancer, oral cancer, tonsillar cancer, breast cancer, etc.

The dosage form of the medicine can be of solvent type or pulvis, etc.

The said solvent has many options, such as cell culture fluid or medium, physiological saline or phosphate buffer, etc.

One or more pharmaceutically acceptable carriers can optionally be added into the said medicine. The said carriers include the conventional diluting agent, absorption promoter, surfactant, etc. in pharmaceutical field.

The method of administration can be isolating the monocytes (Mo) of a patient, infecting or transfecting the Mo with the drug, then inducing the Mo in vitro to be dendritic cells (DC) with antigen presenting function. The said medicine can also be used to infect or transfect DC, but may result in DC having poor ability on antigen capture and processing, and thus unsatisfactory therapeutic effect. The obtained DC can be reinfused to the patient for medical purpose. Alternatively, the cytotoxic-T-lymphocytes (CTL) produced by the stimulation of mature DC expressing mutant HPV-16 E7ₘₘ antigen can be reinfused to patient for better therapeutic effect.

The administration dosage of the said medicine typically is 1-5×10⁶ per time for DC, 1-5×10⁸ per time for CTL, two times per month, And typically three months as a course. The dosage and the treatment course can be adjusted based on actual situation.

In order to enhance therapeutic effect, the said medicine of this invention can also be used in combination with antibiotics, immunostimulants, targeted and chemotherapeutic drugs, etc.

The present invention further provides a method for killing HPV-16-infected cells and HPV-16-E7-positive tumor cells.

The said method comprises the following steps:

1) infecting or transfecting monocytes (Mo) isolated from a patient or dendritic cells (DC) induced from the isolated Mo with the recombinant adeno-associated virus vector carrying HPV-16 E7ₘ antigen gene, AAV/HPV-16 E7ₘ; or treating monocytes isolated from a patient or dendritic cells (DC) induced from the isolated Mo with the products associated with the recombinant adeno-associated virus vector of present invention, to obtain the treated monocytes or dendritic cells, respectively;

2) reinfusing the treated DC of step 1) to the patient to activate immunoreaction in the body of the patient to achieve the purpose of killing the HPV-16-infected cells and HPV-16-E7-positive tumor cells; or mixedly culturing the non-treated T-lymphocytes and the treated DC to stimulate and produce HPV-16-E7-antigen-specific cytotoxic-T-lymphocytes (CTL), the antigen-specific CTL being reinfusing to the patient to kill the HPV-16-infected cells and HPV-16-E7-positive tumor cells; or reinfusing the treated CTL and DC to the patient to kill the HPV-16-infected cells and HPV-16-E7-positive tumor cells.

The said method for killing malignant tumor cells can be applied to clinical treatment, including reinfusing the HPV-16-E7-antigen-specific cytotoxic-T-lymphocytes to the patient, the HPV-16-E7-antigen-specific cytotoxic-T-lymphocytes are produced by mixedly culturing T-lymphocytes naturally produced by the patient and the monocytes-dendritic cells derived from the patient; before mixedly culturing, the monocytes-dendritic cells have been infected or transfected by the recombinant adeno-associated virus vector carrying HPV-16 E7ₘ antigen gene of the present invention or have been treated by the products associated with the recombinant adeno-associated virus vector of the present invention;

alternatively, reinfusing the monocytes-dendritic cells derived from a malignant tumor patient to the patient; before reinfusing, the monocytes-dendritic cells have been infected or transfected by the recombinant adeno-associated virus vector carrying HPV-16 E7ₘ antigen gene of the present invention or have been treated by the products associated with the recombinant adeno-associated virus vector of the present invention;

alternatively, reinfusing the above T-lymphocytes derived from a patient with malignant tumor and monocytes-dendritic cells naturally produced by the patient to the patient; before reinfusing, the T-lymphocytes have been treated by the products associated with the monocytes-dendritic cells inected or transfected by the recombinant adeno-associated virus vector carrying HPV-16 E7ₘ antigen gene of the present invention, the above monocytes-dendritic cells have been infected or transfected by the recombinant adeno-associated virus vector carrying HPV-16 E7ₘ antigen gene of the present invention.

The recombinant adeno-associated virus (rAAV) vector of the present invention can transport the HPV-16 E7ₘ antigen gene carried by it into monocytes-dendritic cell line. Cells carrying HPV-16 E7ₘ antigen gene can be used as effector cells for stimulating immune system (not restricted to T-lymphocytes and B-lymphocytes). It is confirmed by experiments that the dendritic cells infected by the rAAV of the present invention and the cytotoxic-T-lymphocytes induced can effectively kill HPV-16-E7-antigen positive tumor cells or HPV-16 infected cells in vivo, and is nonpathogenic (i.e., non-oncogenic). Therefore, the rAAV vector and products associated with the rAAV vector of the present invention can be used to produce anti-tumor medicines. The present invention is theoretically and practically significant in tumor clinic treatment and application, and possesses wide application prospects.

In order to clarify the content of the invention, which does not mean to limit the invention, the present invention is described in detail in combination with examples as following.

### DESCRIPTION OF THE DRAWINGS

FIG. 1 shows the structural schematic diagram of the recombinant adeno-associated virus vector carrying E7 gene of human papillomavirus type 16 (HPV-16) or mutant HPV-16 E7ₘ gene.

FIG. 2 shows the testing result of agarose gel electrophoresis of the 297 bp HPV-16 E7 DNA obtained from cervical cancer tissue by polymerase chain reaction (PCR).

FIG. 3A-3G show the result of sequences alignments of HPV-16 E7 gene respectively with seven different multiple-site-mutant HPV-16 E7ₘ gene. FIGS. 3A-3G include SEQ ID NO:1 as the “Sbjct” sequence. FIGS. 3A-3G include SEQ ID NOs: 2-8, respectively, as the “Query” sequence.

FIG. 4A shows the endonuclease analysis result of AAV/HPV-16 E7ₘ₅₈ vector.

FIG. 4B shows the endonuclease analysis result of AAV/HPV-16 E7ₘ₉₁ vector.

FIG. 4C shows the endonuclease analysis result of AAV/HPV-16 E7ₘ₉₄ vector.

FIG. 4D shows the endonuclease analysis result of AAV/HPV-16 E7ₘ₂₁ vector, AAV/HPV-16 E7ₘ₂₂ vector and AAV/HPV-16 E7ₘₘ₃ vector.

FIG. 5 shows the flow chart of preparation method for recombinant adeno-associated virus vector (rAAV).

FIG. 6A shows the observation result of oncogenicity of primary cervical epithelial cells infected by three single-site-mutant recombinant adeno-associated virus (AAV/HPV-16 E7ₘ₅₈, AAV/HPV-16 E7ₘ₉₁ and AAV/HPV-16 E7ₘ₉₄).

FIG. 6B shows the observation result of oncogenicity of primary cervical epithelial cells infected by AAV/HPV-16 E7 virus and four multiple-site-mutant recombinant adeno-associated virus (AAV/HPV-16 E7ₘₘ₂₁, AAV/HPV-16 E7ₘₘ₂₂, AAV/HPV-16 E7ₘₘ₂₃ and AAV/HPV-16 E7ₘₘ₃).

FIG. 7 shows the experimental procedure of killing HPV-16-E7 antigen positive cells based on infecting tumor patient's monocytes by AAV/HPV-16 E7ₘ virus.

FIG. 8A shows the detection result of efficiency of AAV/HPV-16 E7ₘ₅₈ with four different promoter, AAV p5, cytomegalovirus promoter, SV40 virus promoter or β-actin promoter, in infecting monocytes (Mo).

FIG. 8B shows the detection result of efficiency of AAV/HPV-16 E7ₘ₉₁ with four different promoter, AAV p5, cytomegalovirus promoter, SV40 virus promoter or β-actin promoter, in infecting monocytes (Mo).

FIG. 8C shows the detection result of efficiency of AAV/HPV-16 E7ₘ₉₄ with four different promoter, AAV p5, cytomegalovirus promoter, SV40 virus promoter or β-actin promoter, in infecting monocytes (Mo).

FIG. 8D shows the detection result of efficiency of four multiple-site-mutant recombinant adeno-associated virus, AAV/HPV-16 E7ₘₘ₂₁, AAV/HPV-16 E7ₘₘ₂₂, AAV/HPV-16 E7ₘₘ₂₃ and AAV/HPV-16 E7ₘₘ₃, in infecting monocytes (Mo).

FIG. 9 shows the flow cytometry detection result of expression level of CD80 and CD60 in dendritic cells infected by AAV/HPV-16 E7ₘ virus and AAV/HPV-16 E7 virus respectively.

FIG. 10 shows the flow cytometry detection result of expression level of IFN-γ in cytotoxic-T-lymphocytes, which were obtained by induction of the dendritic cells infected by AAV/HPV-16 E7ₘ virus and AAV/HPV-16 E7 virus respectively.

FIG. 11A-11G show the ⁵¹Cr assay detection result of cytotoxicity of cytotoxic-T-lymphocytes induced by DC which were infected by AAV/HPV-16 E7ₘ₁ on HPV-16 E7 positive and negative cells in vitro.

FIG. 12A-12D show the changes of serum squamous cell carcinoma antigen (SSC) level and serum keratin 19 antigen (CK19) level of cervical cancer patient after treated by cytotoxic-T-lymphocytes which were obtained by the induction of the dendritic cells infected by recombinant adeno-associated virus (AAV/HPV-16 E7ₘ virus).

FIG. 13 shows the changes of serum keratin 19 antigen (CK19) level of two anal cancer patients after treated by cytotoxic-T-lymphocytes which were obtained by the induction of the dendritic cells infected by recombinant adeno-associated virus (AAV/HPV-16 E7ₘₘ₃ virus).

FIG. 14 shows the changes of serum carcinoembryonic antigen (CEA) level of four penile cancer patients after treated by cytotoxic-T-lymphocytes which were obtained by the induction of the dendritic cells infected by recombinant adeno-associated virus (AAV/HPV-16 E7ₘₘ₃ virus).

### MODE OF CARRYING OUT THE INVENTION

Unless otherwise specified, the method used in the following examples are conventional, and the concrete steps are available in the ((Molecular Cloning: A Laboratory Manual)) (Sambrook, J., Russell, David W., Molecular Cloning: A Laboratory Manual, 3rd edition, 2001, NY, Cold Spring Harbor).

Unless otherwise specified, the “percent concentration” as used herein refers to percent concentration of weight/weight (W/W, with the unit of g/100 mg), percent concentration of weight/volume (W/V, with the unit of g/100 mL), or percent concentration of volume/volume (V/V, with the unit of mL/100 m).

The synthesis of primers and DNA sequencing are completed by Life Technology, USA).

The approach to the acquisition of the various biomaterials described in the examples is merely to provide an experimental approach for achieving specific publicity purposes and should not be limitation of sources of the biomaterials in the present invention. In fact, sources of the biomaterials in the present invention are widely available, and any biological material that does not violate the legal and moral ethic can be replaced in accordance with the prompts in the examples.

The following examples are carried out based on the technical solutions of the present invention. The examples will be helpful to understanding the present invention, while the protection range of the present invention is not limited to the following examples.

The constructure of recombinant adeno-associated virus vector, AAV/HPV-16 E7 and AAV/HPV-16 E7ₘ, will be described in detail in the following examples.

Materials and sources thereof:

A. Four different adeno-associated virus (AAV) vector: the four different AAV vector are respectively named as: pAAV/p5 with AAV p5 promoter, pAAV/CMVp with cytomegalovirus (CMV) promoter, pAAV/SV40p with SV40 virus promoter and pAAV/β-actinp with β-actin promoter. The known adeno-associated virus vector has p5 promoter. To increase the transcription level of the target gene, the p5 promoter in the recombinant adeno-associated virus vector can be replaced with one of the cytomegalovirus (CMV) promoter, β-actin promoter and SV40 viral promoter. The above-mentioned four different adeno-associated virus vector have identical gene structure except for the promoter, i.e., having intact AAV 2-type repeat terminal fragment (TR) sequence at both ends, having an insertion of 9 ribonucleotides (CTGCGCTGG, aim to improve the stability of the recombinant adeno-associated virus (rAAV) and the replication efficiency of the virus) in the 75th nucleotide sequence of TR at both ends, and not having any AAV structural gene (Rep and Cap). The above-mentioned four different adeno-associated virus vectors, pAAV/p5, pAAV/CMVp, pAAV/SV40p and pAAV/β-actinp, were successfully constructed by the inventors of the present patent application (see PCT/CN2008/000835 publication WO2008/128440A1 for the construction method).

B. Human cervical cancer tissue: The human cervical cancer tissue used in experiment was from cervical cancer tissue surgically removed, and confirmed to be HPV-16 antigen positive through immunohistochemistry.

C. Nucleotide primers for gene amplification: designed based on the HPV-16 E7 gene sequence published in American Genbank (NCI gene pool: KC935953). The upstream primer is 5′-ATGCATGGAGATACA-3′, and downstream primer is 5′-TTATTGTTTCTGAGAA-3′.

### Basic Example: Construction of Recombinant Adeno-Associated Virus Vector Carrying the Human Papillomavirus Type 16 (HPV-16) E7 Gene or the Mutant E7ₘ Gene

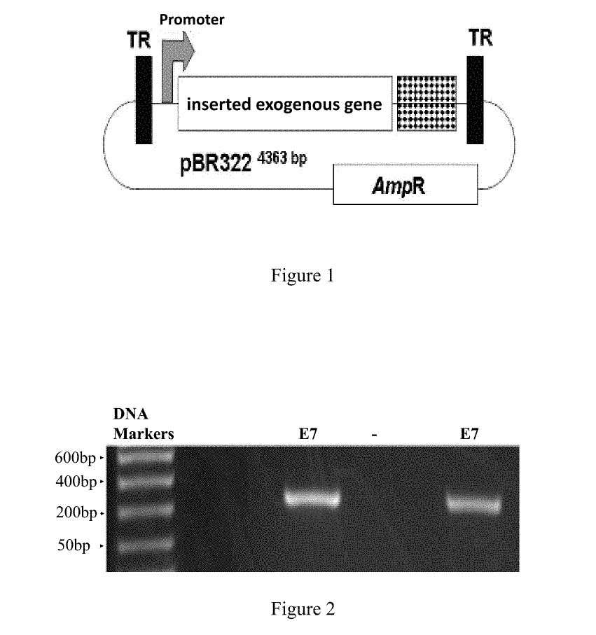

The recombinant adeno-associated virus vector, carrying HPV-16 E7 gene or mutant HPV-16 E7ₘ gene, was respectively constructed by the following method, and the structural schematic diagram thereof is shown in FIG. 1 (the “inserted exogenous gene” are HPV-16 E7 antigen gene or seven HPV-16 E7ₘ antigen gene, and the promoter is any one of AAV p5 promoter, cytomegalovirus (CMV) promoter, β-actin promoter or SV40 virus promoter). The specific process comprises the following steps:

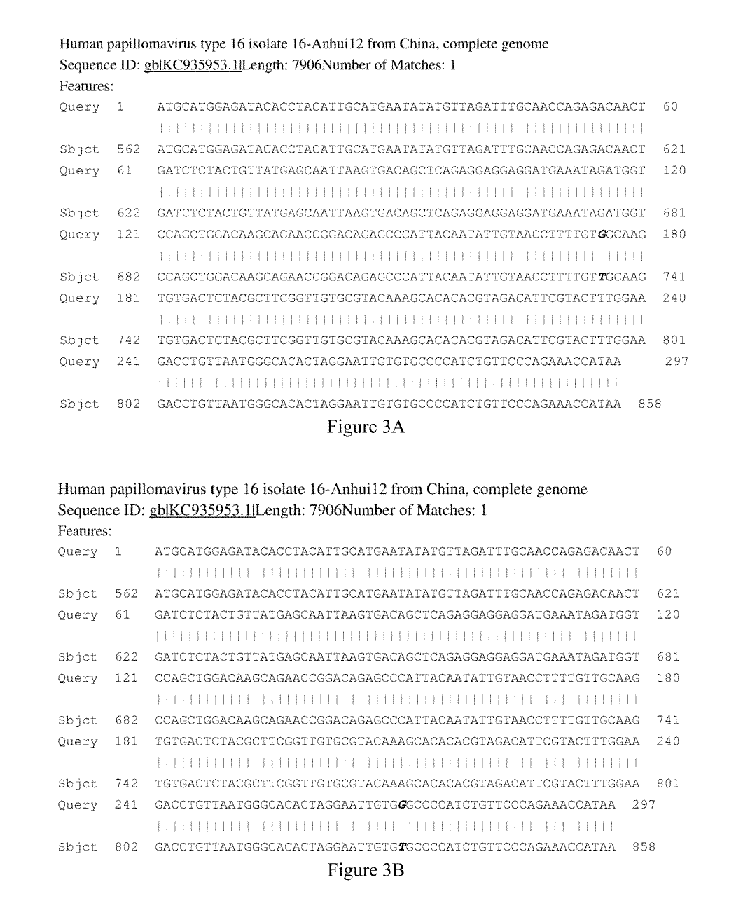

A. Obtaining HPV-16 E7 DNA: The specific method is that the DNAzol reagent (Life Technology, USA) is used according to the instructions. After repeated grinding, the HPV-16-E7 antigen positive cervical cancer tissue was mixed together with 10 mL DNAzol, and centrifugated. After centrifugation, the supernatant was washed twice with 75% ethanol, then absolute ethanol was added, centrifugated, and the precipitate was dissolved in deionized water to adjust the DNA concentration to 100 ng/μL. HPV-16 E7 DNA was amplified by PCR using 2 μL DNA solution as a template under the guidance of the upstream primer 5′-ATGCATGGAGATACA-3′ and the downstream primer 5′-TTATTGTTTCTGAGAA-3′. PCR amplification conditions were as follows: first 94° C. for 4 minutes; then 94° C. for 30 seconds, 60° C. for 35 seconds, and 72° C. for 50 seconds, for 30 cycles; finally 72° C. for 8 minutes. After the reaction, PCR amplification products were analyzed by 1.2% agarose gel electrophoresis. The testing result was shown in FIG. 2. A specific band with the length of 297 bp was obtained and was identical to expection. The band was recovered and purified. And the obtained HPV-16 E7 gene was 297 bp in length. The DNA sequence of obtained HPV-16 E7 gene was determined and the nucleotide sequence thereof was shown in SEQ ID NO: 1 in the Sequence Listing, and it was confirmed that the sequence of PCR-amplified HPV-16 E7 gene was correct;

B. Obtaining the mutant HPV-16 E7ₘ DNA: The specific procedure is described in detail in Examples 1-7;

C. Obtaining the recombinant adeno-associated virus vectors, AAV/HPV-16 E7 and AAV/HPV-16 E7ₘ: The above-obtained HPV-16 E7 DNA fragment and HPV-16 E7ₘ DNA fragment are respectively inserted into the above-mentioned four rAAV: pAAV/p5, pAAV/CMVp, pAAV/SV40p and pAAV/β-actinp by DNA ligation technique. In order to insert the gene fragment, an endonuclease reaction is carried out first, followed by a ligation reaction. The endonuclease reaction system contains 100 ng of plasmid and 50 ng of HPV-16 E7 DNA or HPV-16 E7ₘ DNA fragment; 10 U of restriction endonuclease BamH I and Sal I (purchased from Promega Co. Ltd, USA), 2.5 μL of 10× buffer C and 19.5 μL deionized water; the reaction condition is in water bath at 37° C. for 4 hours. The ligation reaction system contains: 50 ng of digested plasmid, 50 ng of HPV-16 E7 or HPV-16 E7ₘ DNA fragment, 10 IU of T4 DNA ligase (purchased from Promega Co. Ltd, USA), 1.5 μL of 10×T4 DNA ligation buffer and 11.5 μL deionized water; the reaction condition is at 4° C. for 8 hours. Finally, a recombinant adeno-associated virus vector, carrying one of p5 promoter, CMV promoter, SV40 early promoter or β-actin promoter and one of HPV-16 E7ₘ gene or HPV-16 E7 gene, is obtained respectively. The recombinant adeno-associated virus vectors, respectively corresponding to one of the above-mentioned four promoters and respectively carrying one of the above-mentioned seven HPV-16 E7ₘ genes, are collectively called AAV/HPV-16 E7ₘ. And the recombinant adeno-associated virus vectors, respectively corresponding to one of the above-mentioned four promoters and carrying HPV-16 E7 gene, are collectively called AAV/HPV-16 E7;

D. Obtaining plasmids of the recombinant adeno-associated virus vectors, AAV/HPV-16 E7 and AAV/HPV-16 E7ₘ: The ligated AAV/HPV-16 E7ₘ and AAV/HPV-16 E7 are used to transform genetically engineered Escherichia coli DH5a competent cells (purchased from Invitrogen Co. Ltd, USA). Resistance screening is performed using LB agar plates with 100 μg/mL ampicillin and the white colonies are picked, the plasmids of the picked colonies are extracted and purified to obtain plasmids of AAV/HPV-16 E7ₘ and AAV/HPV-16 E7, respectively;

F. Testing the plasmids: Enzyme digestion is made in the obtained AAV/HPV-16 E7ₘ plasmids by use of restriction enzyme BamH I and Sal I (purchased from Promega Co. Ltd, USA) to identify whether the construction is successful. The conditions and methods of the restriction endonuclease reaction are as described above in (Step C of Basic Example).

The construction of the seven AAV/HPV-16 E7ₘ vectors will be described in detail below.

### Example 1: Construction of Recombinant Adeno-Associated Virus Vector AAV/HPV-16 E7ₘ₅₈

The construction method is similar to that in the Basic Example, and the specific operations are:

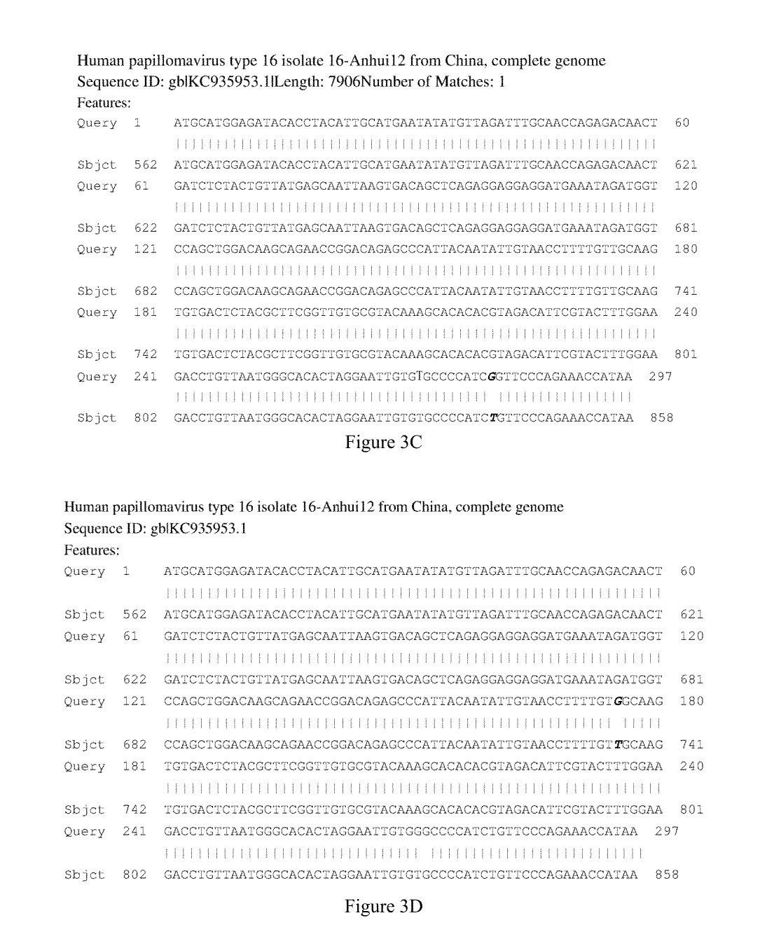

B. Obtaining HPV-16 E7ₘ₅₈ DNA: Site-directed Mutagenesis kit (purchased from Strategeng Co. Ltd, USA) is used according to the instructions thereof. Mutant HPV-16 E7ₘ₅₈ gene without oncogenicity is obtained by replacing thymine (T) in nt175 of the open reading frames of HPV-16 E7 gene (American NCI gene pool: KC935953) with guanine (G), i. e., replacing the “tgc” encoding cysteine (nt175-177) with “ggc” encoding glycine. The obtained HPV-16 E7ₘ₅₈ gene is sequenced, and the sequence thereof is as shown in SEQ ID NO: 2 in the Sequence Listing. The sequences alignments result of HPV-16 E7ₘ₅₈ gene with HPV-16 E7 gene is as shown in FIG. 3A. In FIG. 3A, thymine at position nt736 is replaced with guanine, and “TGC” (nt736-nt738) encoding cysteine is changed to “GGC” encoding glycine, which proves the success of gene mutation and the non-oncogenic HPV-16 E7ₘ₅₈ antigen gene is obtained;

C. Obtaining the recombinant adeno-associated virus vectors, AAV/HPV-16 E7ₘ₅₈: The above-obtained HPV-16 E7ₘ₅₈ DNA fragment is inserted respectively into the pAAV/p5 vector, pAAV/CMVp vector, pAAV/SV40p vector and pAAV/β-actinp vector by DNA ligation technique. Recombinant adeno-associated virus vectors, carrying p5 promoter, CMV promoter, SV40 early promoter or β-actin promoter, are obtained respectively. Four recombinant adeno-associated virus vectors, respectively corresponding to one of the above-mentioned four promoters and carrying HPV-16 E7ₘ₅₈ gene are collectively called AAV/HPV-16 E7ₘ₅₈;

D. Obtaining the plasmids of recombinant adeno-associated virus AAV/HPV-16 E7ₘ₅₈: The ligated AAV/HPV-16 E7ₘ₅₈ is used to transform genetically engineered Escherichia coli DH5a competent cells (purchased from Invitrogen Co. Ltd, USA). Resistance screening is performed using LB agar plates with 100 μg/mL ampicillin and the white colonies are picked, the plasmids of the picked colonies are extracted and purified to obtain AAV/HPV-16 E7ₘ₅₈ plasmids;

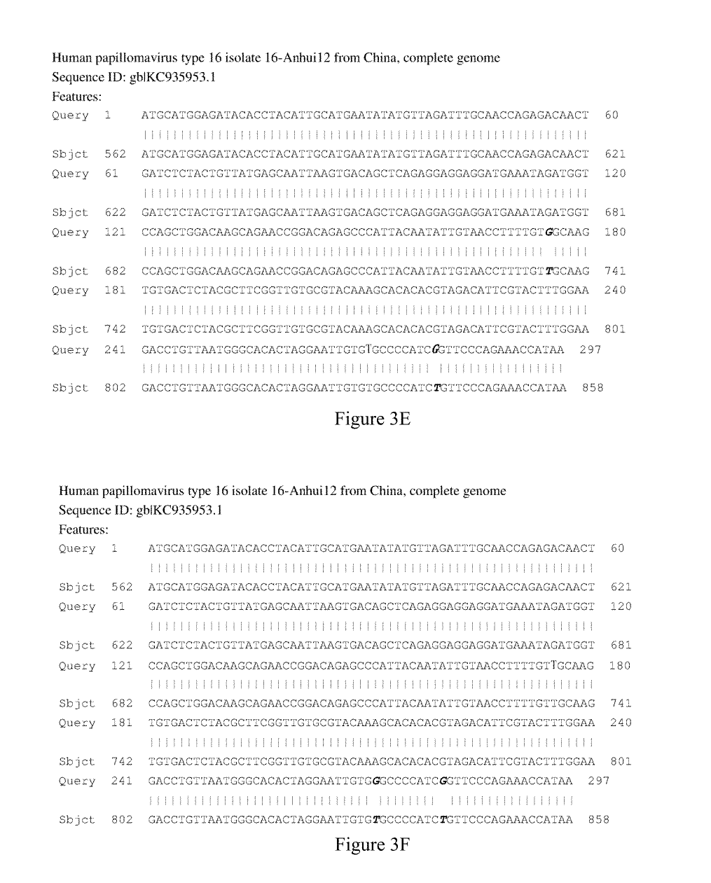

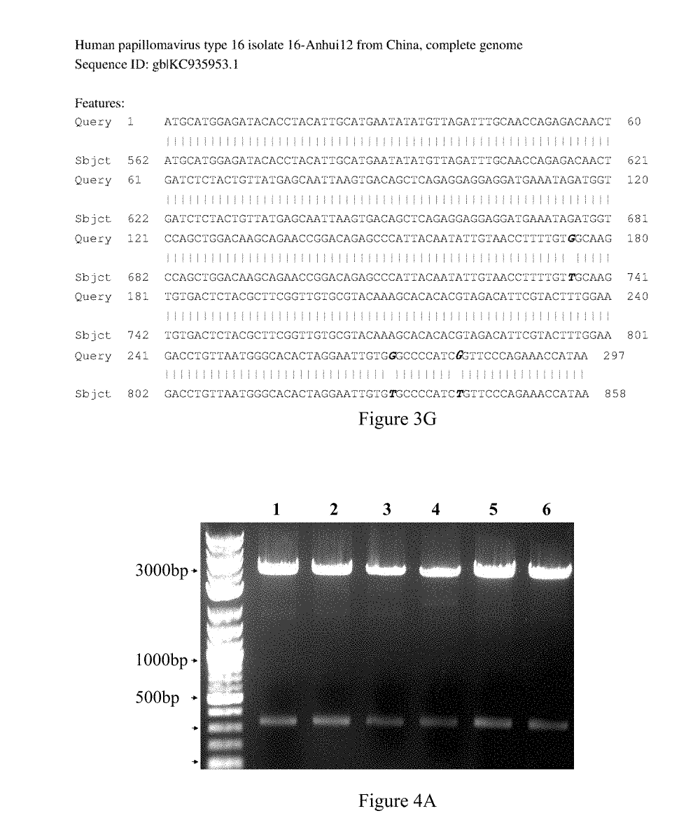

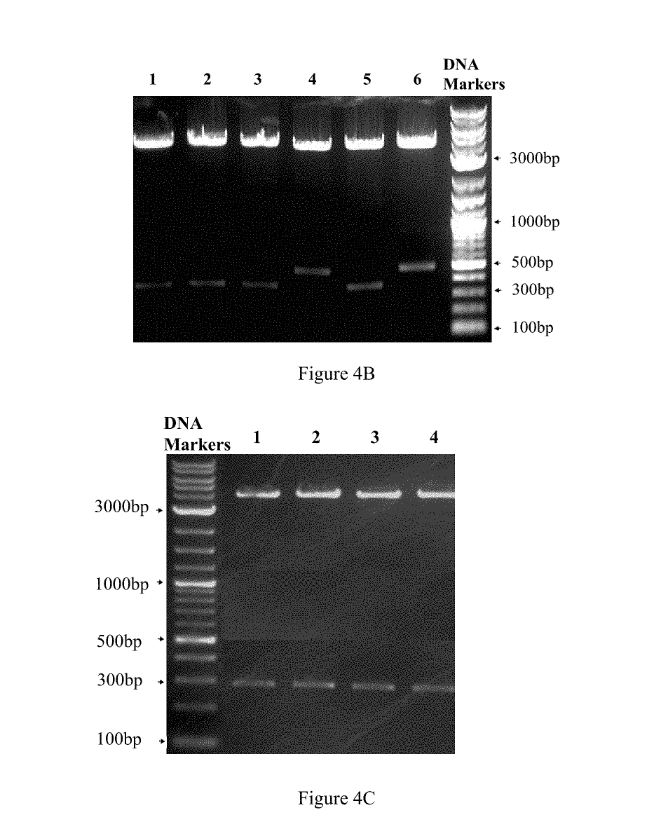

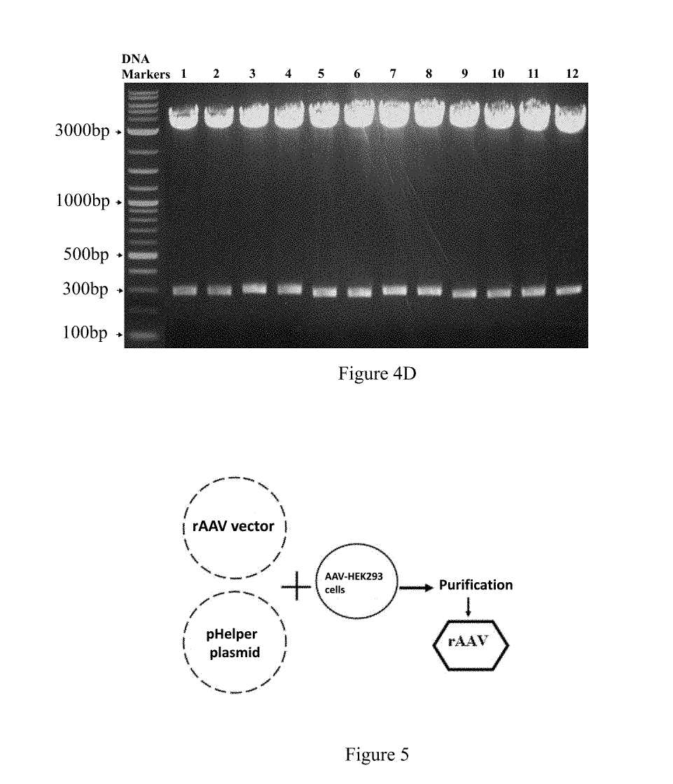

F. Testing the plasmids: Enzyme digestion is made in the obtained AAV/HPV-16 E7ₘ₅₈ plasmids by use of restriction enzyme BamH I and Sal I (purchased from Promega Co. Ltd, USA) to identify whether the construction is successful. The conditions and methods of the restriction endonuclease reaction are as described above in step C of the Basic Example. The endonuclease analysis result of AAV/HPV-16 E7ₘ₅₈ vector is as shown in FIG. 4A (Lane 1-6 are AAV/p5/HPV-16 E7ₘ₅₈, AAV/CMVp/HPV-16 E7ₘ₅₈, AAV/SV40p/HPV-16 E7ₘ₅₈, AAV/β-actinp/HPV-16 E7ₘ₅₈, AAV/p5/HPV-16 E7 and AAV/CMVp/HPV-16 E7, respectively). The result indicates that the recombinant adeno-associated virus vectors carrying HPV-16 E7 gene or mutant HPV-16 E7ₘ₅₈ gene have been successfully constructed.

### Example 2: Construction of Recombinant Adeno-Associated Virus Vector AAV/HPV-16 E7ₘ₉₁

The construction method is similar to that of the Basic Example, and the specific operations are:

B. Obtaining HPV-16 E7ₘ₉₁ DNA: Site-directed Mutagenesis kit (purchased from Strategeng Co. Ltd, USA) is used according to the instructions thereof. Mutant HPV-16 E7ₘ₉₁ gene without oncogenicity is obtained by replacing thymine (T) in nt271 of the open reading frames of HPV-16 E7gene (American NCI gene pool: KC935953) with guanine (G), i. e., replacing the “tgc” encoding cysteine (nt271-273) with “ggc” encoding glycine. The obtained HPV-16 E7ₘ₉₁ gene is sequenced, and the nucleotide sequence thereof is as shown in SEQ ID NO: 3 in the Sequence Listing. The sequences alignments result of HPV-16 E7ₘ₉₁ gene with HPV-16 E7 gene is as shown in FIG. 3B. In FIG. 3B, thymine (T) at position nt832 is replaced with guanine (G), and “TGC” encoding cysteine (nt832-nt834) is changed to “GGC” encoding glycine, which proves the success of the gene mutation and the non-oncogenic HPV-16 E7ₘ₉₁ antigen gene is obtained;

C. Obtaining the recombinant adeno-associated virus vectors, AAV/HPV-16 E7ₘ₉₁: The above-obtained HPV-16 E7ₘ₉₁ DNA fragment is inserted respectively into the pAAV/p5 vector, pAAV/CMVp vector, pAAV/SV40p vector and pAAV/β-actinp vector by DNA ligation technique. Recombinant adeno-associated virus vectors, carrying p5 promoter, CMV promoter, SV40 early promoter or β-actin promoter, are obtained respectively. Four recombinant adeno-associated virus vectors, respectively corresponding to one of the above-mentioned four promoters and carrying HPV-16 E7ₘ₉₁ gene are collectively called AAV/HPV-16 E7ₘ₉₁;

D. Obtaining the plasmids of recombinant adeno-associated virus AAV/HPV-16 E7ₘ₉₁: The ligated AAV/HPV-16 E7ₘ₉₁ is used to transform genetically engineered Escherichia coli DH5a competent cells (purchased from Invitrogen Co. Ltd, USA). Resistance screening is performed using LB agar plates with 100 μg/mL ampicillin, the white colonies are picked, the plasmids of the picked colonies are extracted and purified to obtain AAV/HPV-16 E7ₘ₉₁ plasmids;

F. Testing the plasmids: Enzyme digestion is made in the obtained AAV/HPV-16 E7ₘ₉₁ plasmids by use of restriction enzyme BamH I and Sal I (purchased from Promega Co. Ltd, USA) to identify whether the construction is successful. The conditions and method of the restriction endonuclease reaction are as described above in step C of the Basic Example. The endonuclease analysis result of AAV/HPV-16 E7ₘ₉₁ vector is as shown in FIG. 4B (Lane 1, 2, 3 and 5 are AAV/CMVp/HPV-16 E7ₘ₉₁ plasmids, AAV/SV40p/HPV-16 E7ₘ₉₁ plasmids, AAV/β-actinp/HPV-16 E7ₘ₉₁ plasmids and AAV/p5/HPV-16 E7ₘ₉₁ plasmids, respectively). The result indicates that the recombinant adeno-associated virus vectors carrying HPV-16 E7 gene or mutant HPV-16 E7ₘ₉₁ gene have been successfully constructed.

### Example 3: Construction of Recombinant Adeno-Associated Virus Vector AAV/HPV-16 E7ₘ₉₄

The construction method is similar to that in the Basic Example, and the specific operations are:

B. Obtaining HPV-16 E7ₘ₉₄ DNA: Site-directed Mutagenesis kit (purchased from Strategeng Co. Ltd, USA) is used according to the instructions thereof. Mutant HPV-16 E7ₘ₉₄ gene without oncogenicity is obtained by replacing thymine (T) in nt280 of the open reading frames of HPV-16 E7 gene (American NCI gene pool: KC935953) with guanine (G), i. e., replacing the “tgt” encoding cysteine (nt280-282) with “ggt” encoding glycine. The obtained HPV-16 E7ₘ₉₄ gene is sequenced, and the nucleotide sequence thereof is as shown in SEQ ID NO: 4 in the Sequence Listing and FIG. 3C. The sequences alignments result of HPV-16 E7ₘ₉₄ gene with HPV-16 E7 gene is as shown in FIG. 3C. In FIG. 3C, thymine at position nt841 is replaced with guanine, and “TGT” (nt841-nt843) encoding cysteine is changed to “GGT” encoding glycine, which proves the success of the gene mutation and the non-oncogenic HPV-16 E7ₘ₉₄ antigen gene is obtained;

C. Obtaining the recombinant adeno-associated virus vectors, AAV/HPV-16 E7ₘ₉₄: The above-obtained HPV-16 E7ₘ₉₄ DNA fragment is inserted respectively into the pAAV/p5 vector, pAAV/CMVp vector, pAAV/SV40p vector and pAAV/β-actinp vector by DNA ligation technique. Recombinant adeno-associated virus vectors, carrying p5 promoter, CMV promoter, SV40 early promoter or β-actin promoter, are obtained respectively. Four recombinant adeno-associated virus vectors, corresponding to one of the above-mentioned four promoters respectively and carrying HPV-16 E7ₘ₉₄ gene are collectively called AAV/HPV-16 E7ₘ₉₄;

D. Obtaining the plasmids of recombinant adeno-associated virus AAV/HPV-16 E7ₘ₉₄: The ligated AAV/HPV-16 E7ₘ₉₄ is used to transform genetically engineered Escherichia coli DH5a competent cells (purchased from Invitrogen Co. Ltd, USA). Resistance screening is performed using LB agar plates with 100 μg/mL ampicillin, the white colonies are picked, and the plasmids of the picked colonies are extracted and purified to obtain AAV/HPV-16 E7ₘ₉₄ plasmids;

F. Testing the plasmids: Enzyme digestion is made in the obtained AAV/HPV-16 E7ₘ₉₄ plasmids by use of restriction enzyme BamH I and Sal I (purchased from Promega Co. Ltd, USA) to identify whether the construction is successful. The conditions and method of the restriction endonuclease reaction are as described above in step C of the Basic Example. The endonuclease analysis result of AAV/HPV-16 E7ₘ₉₄ vector is as shown in FIG. 4B (Lane 1-4 are AAV/p5/HPV-16 E7ₘ₉₄ plasmids, AAV/CMVp/HPV-16 E7ₘ₉₄ plasmids, AAV/SV40p/HPV-16 E7ₘ₉₄ plasmids and AAV/β-actinp/HPV-16 E7ₘ₉₄ plasmids respectively). The results shows that HPV-16 E7ₘ₉₄ gene is inserted into AAV vectors carrying different promoters respectively, the recombinant adeno-associated virus vectors carrying mutant HPV-16 E7ₘ₉₄ gene have been successfully constructed.

### Example 4: Construction of Multiple-Site-Mutant Recombinant Adeno-Associated Virus Vector AAV/HPV-16 E7ₘₘ

The construction method is similar to that in the Basic Example, and the specific operations are:

B. Obtaining HPV-16 E7ₘ DNA with two or three mutation sites: the HPV-16 E7ₘ genes with two or three mutation sites are collectively called HPV-16 E7ₘₘ in the present invention;

(1) HPV-16 E7ₘₘ₂₁: the first HPV-16 E7 antigen gene with two mutation sites is obtained and named as HPV-16 E7ₘₘ₂₁ after replacing thymine (T) in nt175 of the open reading frames of HPV-16 E7 gene (American NCI gene pool: KC935953) with guanine (G), i. e., replacing the “tgc” encoding cysteine (nt175-177) with “ggc” encoding glycine, and replacing thymine (T) in nt271 of the open reading frames of HPV-16 E7 gene with guanine (G), i. e., replacing the “tgc” encoding cysteine (nt271-273) with “ggc” encoding glycine. The obtained HPV-16 E7ₘₘ₂₁ gene is sequenced, and the nucleotide sequence thereof is as shown in SEQ ID NO: 5 in the Sequence Listing. The sequences alignments result of HPV-16 E7ₘₘ₂₁ gene with HPV-16 E7 gene is as shown in FIG. 3D (italic in nt841 and nt832 are the mutations). FIG. 3D shows that nucleotide sequences in nt736-nt738 and nt832-nt834 of wild-type HPV-16 E7 are both “TGC” encoding cysteine. While the DNA sequencing result shows that the above-mentioned two triplet codons in the obtained HPV-16 E7ₘₘ₂₁ gene have been both changed to “GGC” encoding glycine, which proves the success of the gene mutation;

(2) HPV-16 E7ₘₘ₂₂: the second HPV-16 E7 antigen gene with two mutation sites is obtained and named as HPV-16 E7ₘₘ₂₂ after replacing thymine (T) in nt175 of the open reading frames of HPV-16 E7 gene (American NCI gene pool: KC935953) with guanine (G), i. e., replacing the “tgc” encoding cysteine (nt175-177) with “ggc” encoding glycine; and replacing thymine (T) in nt280 of the open reading frames of HPV-16 E7 gene with guanine (G), i. e., replacing the “tgt” encoding cysteine (nt280-282) with “ggt” encoding glycine. The obtained HPV-16 E7ₘₘ₂₂ gene is sequenced, and the nucleotide sequence thereof is as shown in SEQ ID NO: 6 in the Sequence Listing. The sequences alignments result of HPV-16 E7ₘₘ₂₂ gene with HPV-16 E7 gene is as shown in FIG. 3E (italic in nt736 and nt841 are the mutations). FIG. 3E shows that nucleotide sequences in nt736-nt738 and nt841-nt843 of wild-type HPV-16 E7 are “TGC” and “TGT” respectively, both encoding cysteine. While the DNA sequencing result shows that the above-mentioned two triplet codons in the obtained HPV-16 E7ₘₘ₂₂ gene is respectively changed to “GGC” and “GGT” encoding glycine, which proves the success of the gene mutation;

(3) HPV-16 E7ₘₘ₂₃: the third HPV-16 E7 antigen gene with two mutation sites is obtained and named as HPV-16 E7ₘₘ₂₃ after replacing thymine (T) in nt271 of the open reading frames of HPV-16 E7gene (American NCI gene pool: KC935953) with guanine (G), i. e., replacing the “tgc” encoding cysteine (nt271-273) with “ggc” encoding glycine, and replacing thymine (T) in nt280 of the open reading frames of HPV-16 E7 gene with guanine (G), i. e., replacing the “tgt” encoding cysteine (nt280-282) with “ggt” encoding glycine. The obtained HPV-16 E7ₘₘ₂₃ gene is sequenced, and the nucleotide sequence thereof is as shown in SEQ ID NO: 7 in the Sequence Listing. The sequences alignments result of HPV-16 E7ₘₘ₂₃ gene with HPV-16 E7 gene is as shown in FIG. 3F (italic in nt832 and nt841 are the mutations). FIG. 3E shows that nucleotide sequences in nt832-nt834 and nt841-nt843 of the wild-type HPV-16 E7 are “TGC” and “TGT” respectively, both encoding cysteine. While the DNA sequencing result shows that the above-mentioned two triplet codons in the obtained HPV-16 E7ₘₘ₂₃ gene have been respectively changed to “GGC” and “GGT” encoding glycine, which proves the success of the gene mutation;

(4) HPV-16 E7ₘₘ₃: the HPV-16 E7 antigen gene with three mutation sites is obtained and named as HPV-16 E7ₘₘ₃ after using the above obtained HPV-16 E7ₘₘ₂₁ DNA with two mutation sites as template, and the thymine (T) in nt280 of HPV-16 E7ₘₘ₂₁ DNA is further replaced with guanine (G), i. e., “tgt” (nt280-282) is replaced with “ggt”. The obtained HPV-16 E7ₘₘ₃ gene is sequenced, and the nucleotide sequence thereof is as shown in SEQ ID NO: 8 in the Sequence Listing. The sequences alignments result of HPV-16 E7ₘₘ₃ gene with HPV-16 E7 gene is as shown in FIG. 3G (italic in nt736, nt832 and nt841 are the mutations). FIG. 3E shows that nucleotide sequences in nt736-nt738, nt832-nt834 and nt841-nt843 of the wild-type HPV-16 E7 are “TGC”, “TGC” and “TGT” respectively, all encoding cysteine. While the DNA sequencing result shows that the above-mentioned three triplet codons in the obtained HPV-16 E7ₘₘ₃ gene have been respectively changed to “GGC”, “GGC” and “GGT”, all encoding glycine, which proves the success of the gene mutation;

C. Obtaining the multiple-site-mutant recombinant adeno-associated virus vectors, AAV/HPV-16 E7ₘₘ: One of the above-obtained HPV-16 E7ₘₘ DNA fragments (three with 2 mutation sites and one with 3 mutation sites) is inserted into one of the four rAAV vectors: pAAV/p5 vector, pAAV/CMVp vector, pAAV/SV40p vector and pAAV/β-actinp vector by DNA ligation technique. The recombinant adeno-associated virus vectors with HPV-16 E7ₘₘ gene, and respectively carrying p5 promoter, CMV promoter, SV40 early promoter or β-actin promoter are obtained. The recombinant adeno-associated virus vectors carrying HPV-16 E7ₘₘ gene (carrying one of HPV-16 E7ₘ₂₁, HPV-16 E7ₘ₂₂, HPV-16 E7ₘ₂₃ and HPV-16 E7ₘ₃), respectively corresponding to one of the above-mentioned four promoters, are collectively called AAV/HPV-16 E7ₘₘ;

D. Obtaining the plasmids of the recombinant adeno-associated virus vectors AAV/HPV-16 E7ₘₘ: The ligated AAV/HPV-16 E7ₘₘ are respectively used to transform genetically engineered Escherichia coli DH5a competent cells (purchased from Invitrogen Co. Ltd, USA), resistance screening is performed by using LB agar plates with 100 μg/mL, the white colonies are picked, the plasmids of the picked colonies are extracted and purified to obtain AAV/HPV-16 E7ₘₘ plasmids;

F. Testing the plasmids: enzyme digestion is made in the obtained AAV/HPV-16 E7ₘₘ plasmids and AAV/HPV-16 E7 plasmids by use of BamH I and Sal I (purchased from Promega Co. Ltd, USA) to identify whether the construction is successful. The conditions and method of the restriction endonuclease reaction are as described above in step C of the Basic Example. The endonuclease analysis results of AAV/HPV-16 E7ₘₘ vectors are as shown in FIG. 4D (taking AAV/CMVp/HPV-16 E7ₘₘ, AAV/SV40p/HPV-16 E7ₘₘ and AAV/β-actinp/HPV-16 E7ₘₘ as examples, Lane 1-4, 5-8 and 9-12 are AAV/HPV-16 E7ₘ₂₁ carrying CMV promoter, SV40 early promoter or β-actin promoter, AAV/HPV-16 E7ₘ₂₂ carrying CMV promoter, SV40 early promoter or β-actin promoter, SV40 virus promoter or β-actin promoter, AAV/HPV-16 E7ₘ₂₃ carrying CMV promoter, SV40 early promoter or β-actin promoter and AAV/HPV-16 E7ₘₘ₃ carrying CMV promoter, SV40 early promoter or β-actin promoter respectively). The results show that the recombinant adeno-associated virus vectors carrying HPV-16 E7 gene or multiple-site-mutant HPV-16 E7ₘₘ gene have been successfully constructed.

### Example 5: Preparation of Recombinant Adeno-Associated Virus (rAAV) and Measurement of Virus Titer

Materials and sources thereof:

A. recombinant adeno-associated virus vectors carrying the HPV-16 E7ₘ and HPV-16 E7 antigen genes constructed in Examples 1-4 (AAV/HPV-16 E7ₘ and AAV/HPV-16 E7).

B. Helper plasmids containing Rep gene and Cap gene of AAV, pHelper: constructed by the inventors of the present invention, Liu Yong et al. (Liu, Y., Santin A D., Mane M., Chiriva-Internati, M., Parham G P., Ravaggi A., and Hermonat, P. L. Transduction and Utility of the Granulocyte-Macrophage Colony-Stimulating Factor Gene into Monocytes and Dendritic Cells by Adeno-Associated Virus. Journal of Interferon and Cytokine Research. 20:21-30.2000).

C. AAV-HEK293 cells containing adenovirus genes (E1, E2A, E4, VAI and VAII genes) integrated on the chromosomes of cells, AAV-HEK293 cells: constructed by the inventors of the present invention, Liu Yong et al. (Liu, Y., Santin A D., Mane M., Chiriva-Internati, M., Parham G P., Ravaggi A., and Hermonat, P. L. Transduction and Utility of the Granulocyte-Macrophage Colony-Stimulating Factor Gene into Monocytes and Dendritic Cells by Adeno-Associated Virus. Journal of Interferon and Cytokine Research. 20:21-30.2000).

D. Liposome transfection reagent, Lipofectin: purchased from Life Technology, USA.

E. DMEM medium and fetal bovine serum (or calf serum): purchased from Cellgro, USA.

F. PCR DIG labeling kit and DIG hybridization detection kit: purchased from Roche, Switzerland

G. DNA copy number standards: 10¹² copies/μL to 10⁶ copies/μL, respectively, purchased from Promega, USA.

1. Preparation of Recombinant Adeno-Associated Virus (rAAV)

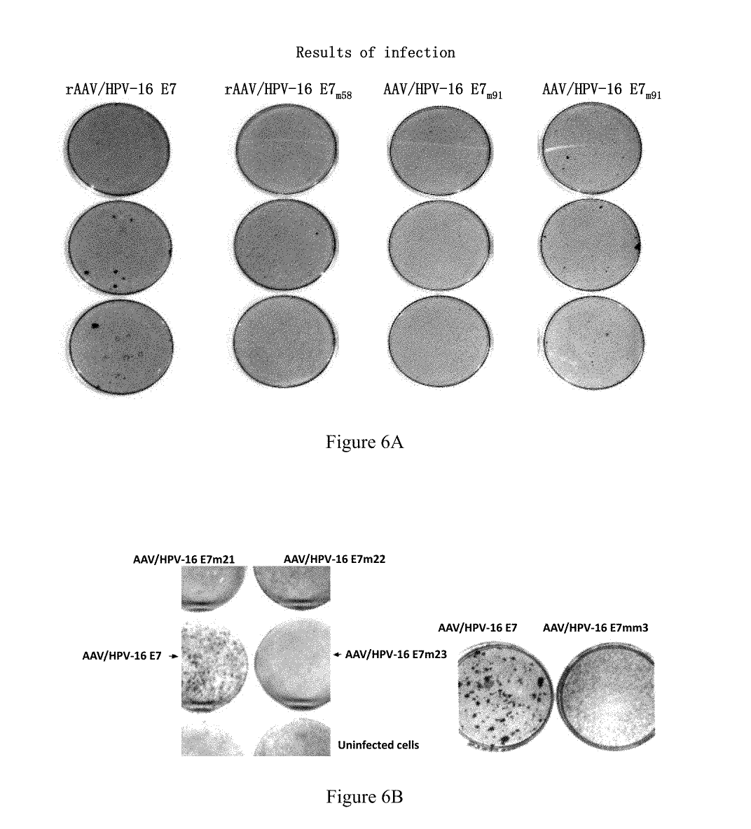

The flow charts of preparation method for the recombinant adeno-associated virus (rAAV) carrying the HPV-16 E7ₘ gene (AAV/HPV-16 E7ₘ) and rAAV carrying the HPV-16 E7 gene (AAV/HPV-16 E7) are shown in FIG. 5. As shown in FIG. 5, the recombinant adeno-associated virus (rAAV) is prepared as following: Taking preparation of virus in a 10.0 cm cell culture dish as an example, when the AAV-HEK293 cells are grown in a carbon dioxide incubator to about 70% of the culture dish area, the following operations are carried out:

A. operate following the Lipofectin instructions: mix together 1.0 μg of rAAV, 1.0 μg of pHelper plasmids, 4.0 μL of Lipofectin and 50.0 μL of DMEM medium containing 5% fetal bovine serum (or calf serum), and take the mixture to stand at room temperature for 20 minutes;

B. add the mixture to a cell culture dish and incubate in a carbon dioxide incubator;

C. after 72 hours incubation, collect all the cells and culture medium in the culture dish;

D. after 1 minute of vigorous oscillation, the collected substance is centrifuged and collect the supernatant, i.e., the rAAV virus solution; and

E. sterilize the collected rAAV virus solution by filtration.

2. Virus Titer Measurement of Recombinant Adeno-Associated Virus (rAAV)

The virus titer of the rAAV obtained in step 1 is measured by a conventional dot-blot hybridization method, and the specific method comprises the following steps:

A. extracting DNA of rAAV particles by conventional DNA phenol-chloroform extraction method;

B. placing nylon membrane in a dot blotter, adding the alkaline-denatured DNA of rAAV particles and the DNA copy number standards, followed by evacuation;

C. removing and drying the nylon membrane, then immobilizing by UV;

D. preparing the DIG-labeled specific probe by PCR DIG labeling kit (following the PCR DIG labeling kit instructions), the DNA probe is a specific probe for the HPV-16 E7 gene which is the HPV16-E7 DNA obtained in the Basic Example; after amplification of the PCR products, analyzing the PCR amplification products by 1.2% agarose gel electrophoresis, detecting the PCR amplification products under UV light, the positive band showing the probe is successfully labeled; and

E. hybridizing the DNA of each rAAV particles in hybridization oven by using DIG hybridization detection kit and with reference to the kit instructions.

The experiment results shows that the virus titers of all rAAV virus (seven AAV/HPV-16 E7ₘ viruses and one AAV/HPV-16 E7 virus) varied from 10¹¹ copies/mL to 10¹⁴ copies/mL, which indicates that the obtained rAAV viruses have high virus titer and can be used for research and clinical practice.

### Example 6: Observation on the Oncogenicity of Primary Cervical Epithelial Cells Infected by Recombinant Adeno-Associated Virus, AAV/HPV-16 E7ₘ and AAV/HPV-16 E7

Materials and sources:

A. rAAV viruses: AAV/HPV-16 E7ₘ virus and AAV/HPV-16 E7 virus obtained in Basic Example and Example 1-4.

B. Keratinocyte-SCF cell culture medium: purchased from Life Technology, USA.

C. Primary cervical epithelial cells: obtained by conventional method from normal cervical epithelial tissue.

Observation on the Oncogenicity

Primary cervical epithelial cells are placed in 10.0 cm cell culture dishes and 10 mL of Keratinocyte-SCF cell culture medium is immediately added therein, and then cultured in a carbon dioxide incubator at 37° C. After the cells are completely adhered, the culture dishes are removed. 7 mL of culture medium is removed, the recombinant adeno-associated virus AAV/HPV-16 E7ₘ or AAV/HPV-16 E7 is added according to the dose of 100 MOI, and the culture dishes are reseted in the carbon dioxide incubator. After 8 hours of incubation, the culture plates are taken out and the culture medium is replaced with 10 mL of fresh Keratinocyte-SCF cell culture medium. Then the culture dishes are reseted in the carbon dioxide incubator at 37° C. and the culture medium is changed every 2 days. The morphological changes of cells are regularly observed twice a day until the cells become neoplastic.

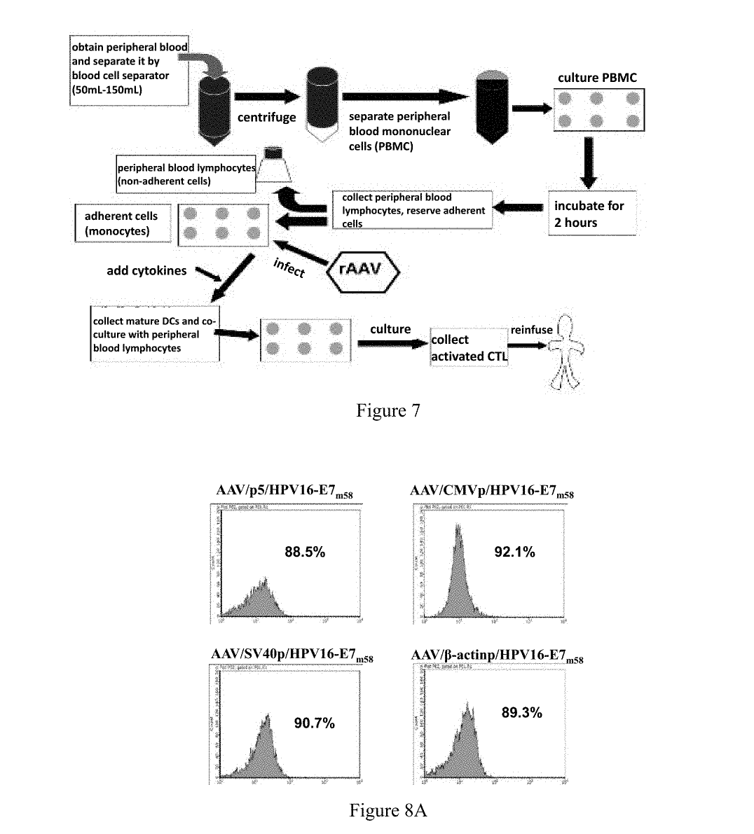

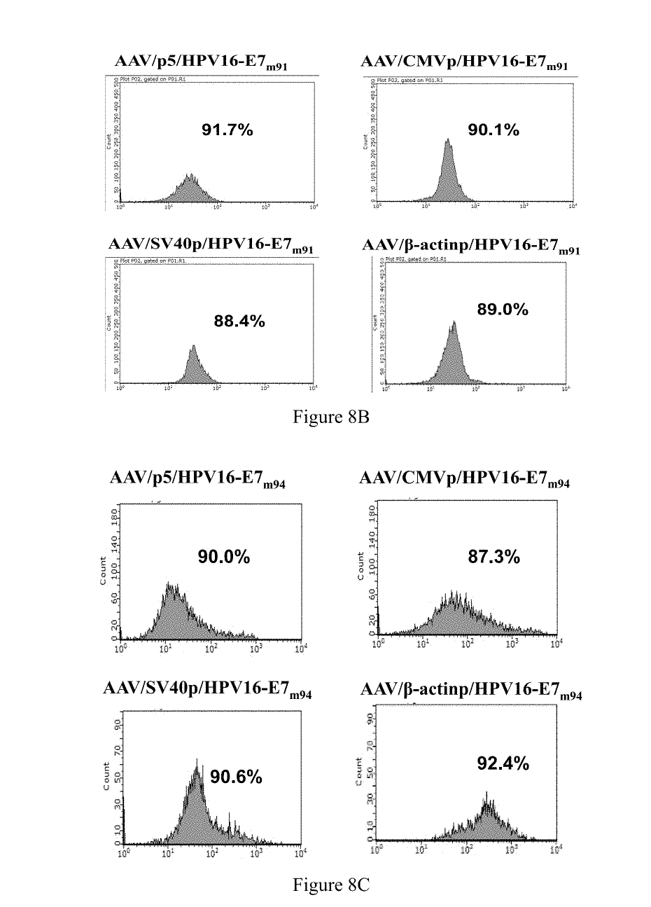

FIGS. 6A and 6B show the observation result of oncogenicity of primary cervical epithelial cells infected by three single-site-mutant recombinant adeno-associated virus (AAV/HPV-16 E7ₘ₅₈, AAV/HPV-16 E7ₘ₉₁ and AAV/HPV-16 E7ₘ₉₄), four multiple-site-mutant recombinant adeno-associated virus (AAV/HPV-16 E7ₘₘ, represented by rAAV carrying CMV promoter) and AAV/HPV-16 E7 virus. The results show that the cells infected by AAV/HPV-16 E7ₘ virus remain normal, which indicate that the AAV/HPV-16 E7ₘ virus are non-oncogenic. The cells infected by AAV/HPV-16 E7 become distinctly neoplastic, which indicate that the AAV/HPV-16 E7 virus is oncogenic. The results show that the rAAVs are all expressed in the cells, but the HPV-16 E7 antigen protein leads to cell neoplasia, while the HPV-16 E7ₘ antigen protein does not lead to cell neoplasia.

### Examples 7: Tumor-Killing Experiment of Monocytes-Derived Dendritic Cells by Tumor Antigen Introducing

Materials and sources:

A. rAAV viruses: AAV/HPV-16 E7ₘ viruses obtained in Example 1-4 and AAV/HPV-16 E7 virus obtained in Basic Example.

B. AIM-V cell culture medium: purchased from Life Technology, USA.

C. Cytokines: granulocyte-macrophage colony stimulating factor (GM-CSF) and Interleukin 2, 4 and 7, all purchased from R&D, USA.

D. HPV-16-E7-positive Cells: isolated from tumor tissue or obtained from American Type Culture Collection (ATCC), including cervical cancer cells, breast cancer cells, penile cancer cells, anal cancer cells and oral cancer cells.

E. HPV-16-E7-negative Cells: isolated from normal human tissue or obtained from American Type Culture Collection (ATCC), including lung, breast, liver and kidney epithelial cells.

1. Tumor-Killing Experiment

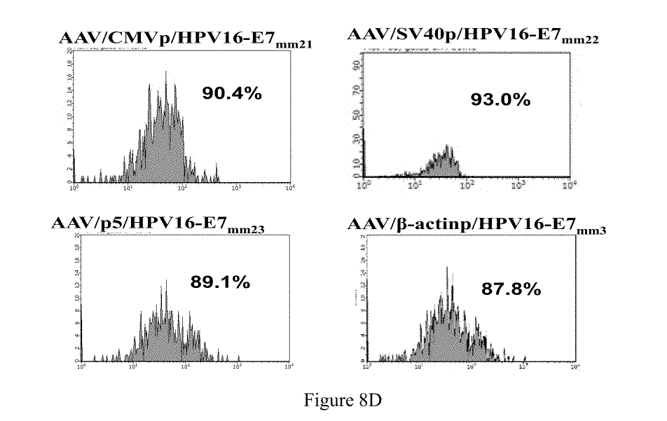

FIG. 7 shows the experiment procedure of killing HPV-16-E7 antigen positive cells based on infecting tumor patient's monocytes by AAV/HPV-16 E₇ₘ. As shown in FIG. 7, the whole experiment process of killing HPV-16-E7 antigen positive cells based on infecting tumor patient's monocytes by rAAV virus carrying HPV-16 E7 or HPV-16 E7ₘ antigen gene (AAV/HPV-16 E7 virus or AAV/HPV-16 E7ₘ virus) in the present invention comprises the following steps:

A. according to the conventional method, the peripheral blood mononuclear cells (PBMC) are obtained by blood cell separator (or lymphocyte separation medium) from 50-150 mL peripheral blood of a tumor patient, after mixed with AIM-V medium, the cells are added to the cell culture flask, and placed in constant temperature carbon dioxide incubator at 37° C. for 2 hours;

B. the suspended cells are removed, and the adherent cells (i. e., monocytes) are reserved; the suspended cells (i. e., peripheral blood lymphocytes) are mixed with the IM-V medium and kept incubation;

C. about 100 MOI of rAAV virus and GM-CSF (600 IU/mL) are added, and incubation is continued for another 4 hours;

D. the old medium is removed and fresh AIM-V medium containing GM-CSF and IL-4 (600 IU/mL) is supplemented, incubation is continued;

E. after 5 days of incubation, the mature dendritic cells (DC) is collected and mixed with the cultured peripheral blood lymphocytes, IL-2 (10 IU/mL) is added and incubation is continued; and

F. after 7-9 days of incubation, the activated cytotoxic-T-lymphocytes (CTL) are collected and detected.

2. Detection of Dendritic Cells (DC) and Cytotoxic-T-Lymphocytes (CTL)

A. Detection Experiment of Efficiency of rAAV in Infecting Peripheral Blood Monocytes (Mo)

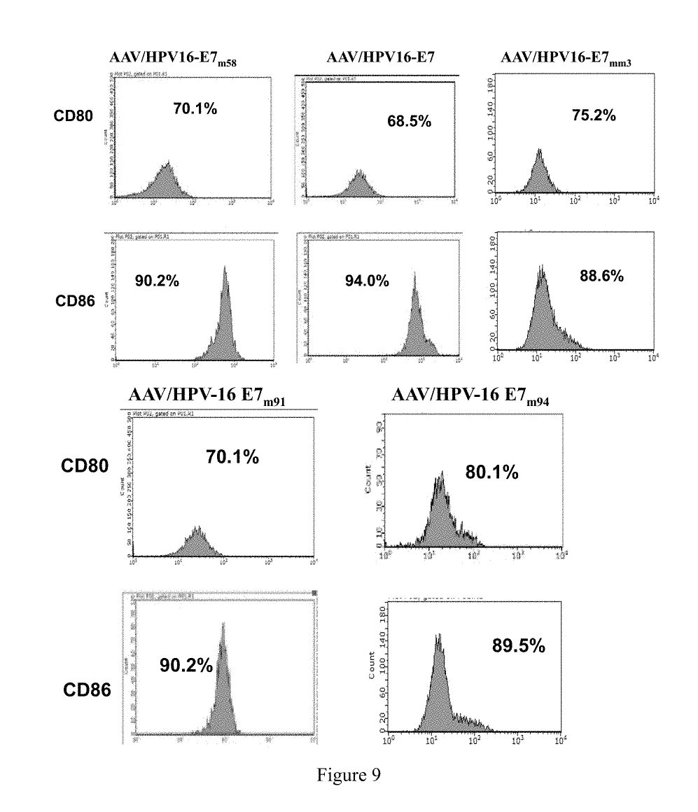

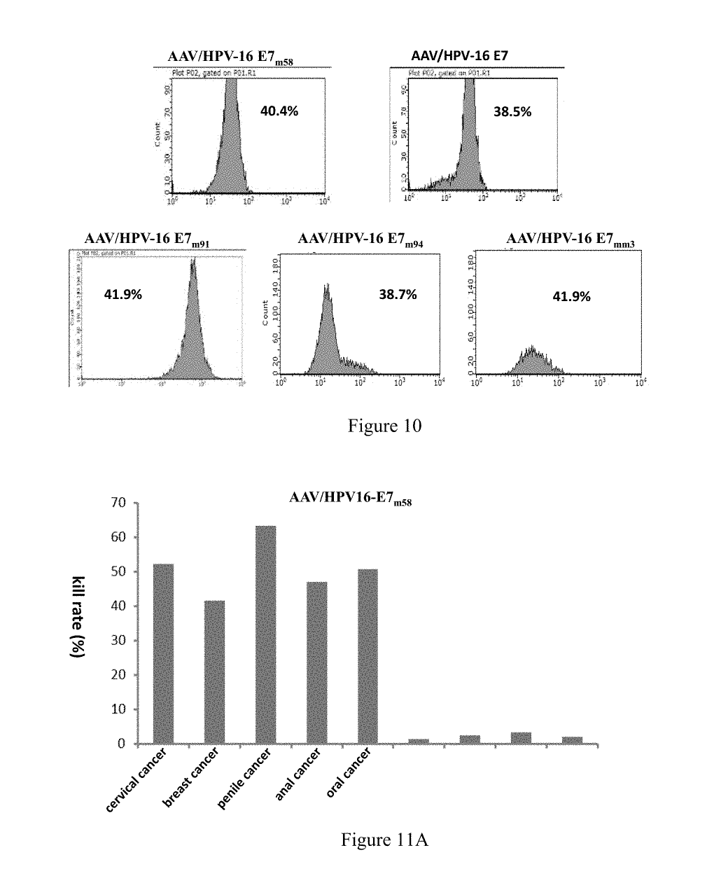

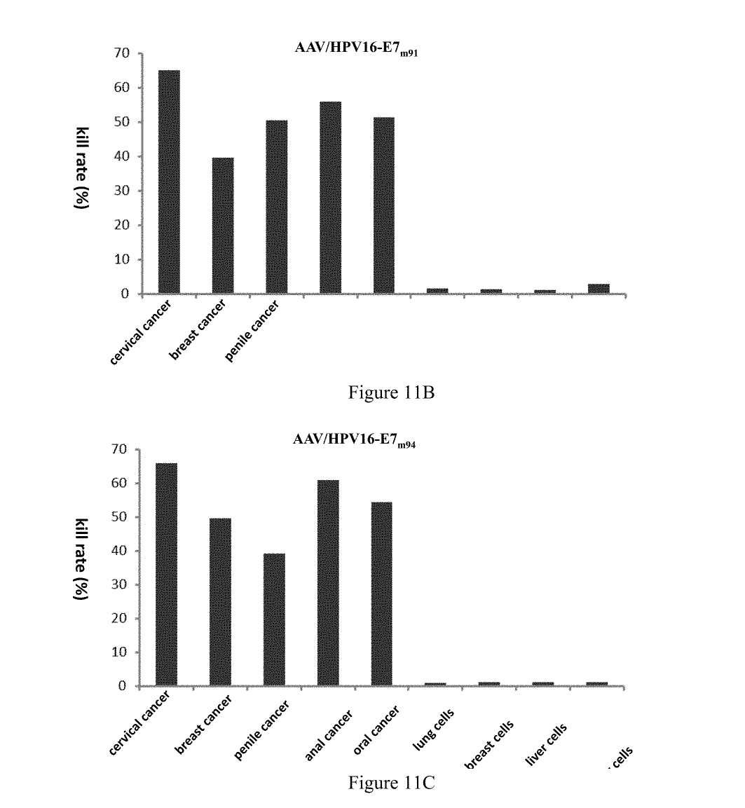

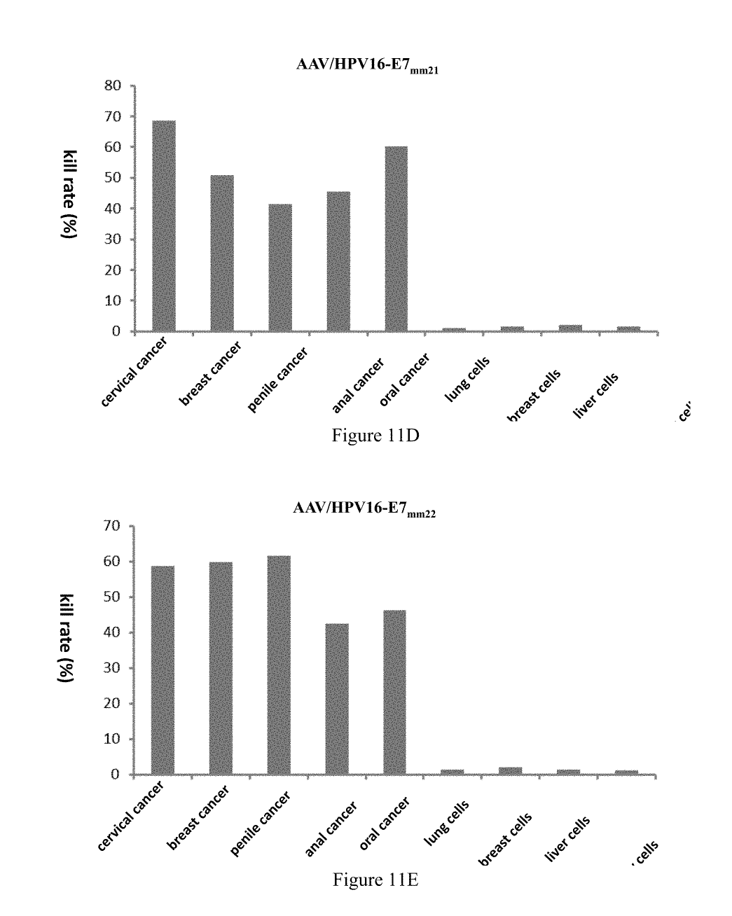

By use of the conventional fluorescent antibody staining method, the mononuclear cells (Mo) infected by the rAAV in the present invention or the immature dendritic cells obtained in step 1 are labeled with the HPV-16-E7-specific fluorescent antibody (purchased from BD, USA). And the number of positive cells is detected by flow cytometer. The detection results of efficiency of rAAV in infecting peripheral blood monocytes (Mo) are shown in FIG. 8A-8D, wherein the efficiency of AAV/HPV-16 E7ₘ viruses carrying four different promoters (p5, CMVp, SV40p and β-actinp) and AAV/HPV-16 E7 virus in infecting peripheral blood monocytes (Mo) are about 90%, i. e., approximately 90% of Mo can be infected by rAAV virus, which proves the high infection efficiency of the rAAV of the present invention.

B. Detection of CD Molecule Level in Dendritic Cells (DC)

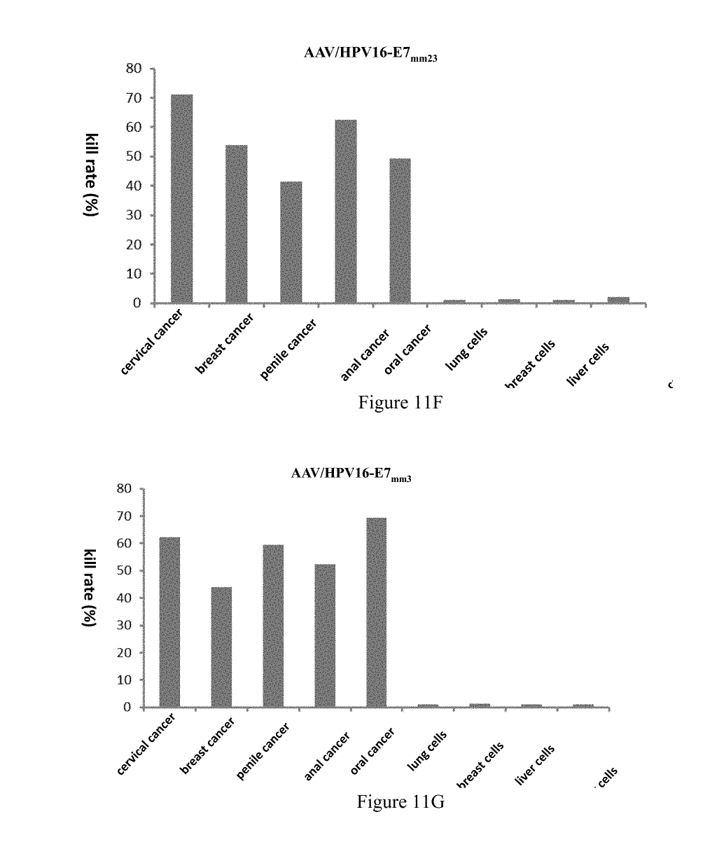

The expression level of CD80 and CD86 in DC is positively correlated with the function of DC. The expression level of CD80 and CD86 in DC obtained in step 1 is detected by the same detection method as that in step A, i. e., fluoresce-labeled antibodies (purchased from BD, USA) against the two CD molecules to detect respectively are used. The detection results of CD80 and CD86 expression level in DC respectively infected by AAV/HPV-16 E7ₘ viruses and rAAV/HPV-16 E7 virus are shown in FIG. 9 (rAAV carrying SV40 early promoter as the representative for AAV/HPV-16 E7ₘ₉₁ viruses, and rAAV carrying CMV promoter as the representative for AAV/HPV-16 E7ₘ₉₄ viruses, AAV/HPV-16 E7ₘ₅₈ viruses and AAV/HPV-16 E7ₘₘ₃ viruses). The flow cytometry detection result of expression level of CD80 and CD60 in DC infected by the rAAV carrying CMV promoter, AAV/HPV-16 E7ₘ and AAV/HPV-16 E7 shows that both CD80 and CD86 can be expressed in high level, which indicates that the DC induced by monocytes which are infected by rAAV carrying HPV-16 E7 or HPV-16 E7ₘ antigen gene in the present invention have strong ability to stimulate the cellular immune response.

C. Detection of Expression Level of Interferonγ (IFN-γ) in Cytotoxic-T-Lymphocytes (CTL)

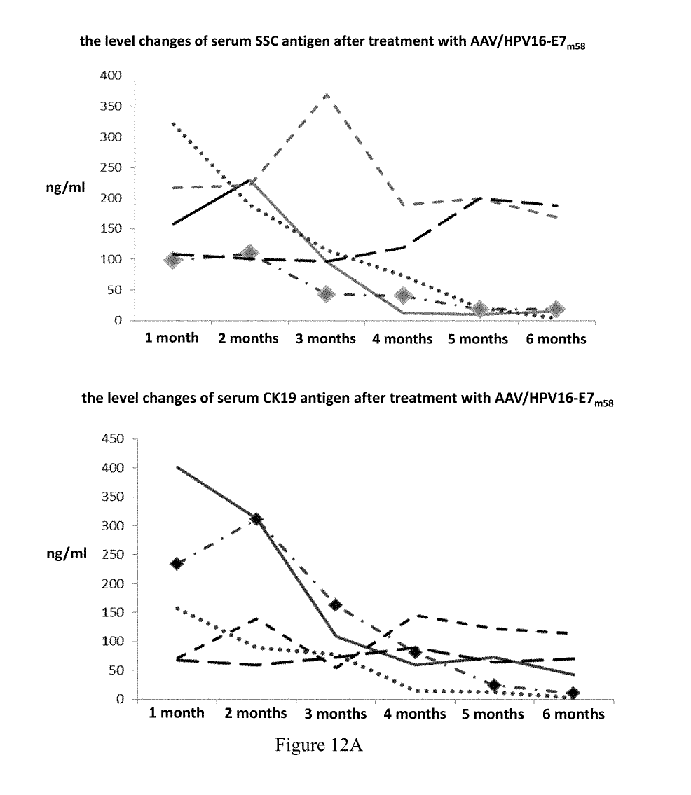

The function of CTL and its ability to kill tumor cells are positively correlated with the expression level of IFN-γ. The expression level of IFN-γ in CTL induced by DC which are infected by rAAV of the present invention is detected by the similar detection method as that in step A. DCs are mixedly cultured with peripheral blood lymphocytes, and the cells are harvested after incubation. The cells are fluorescently labeled by the traditional intracellular cytokine staining method, and the antibody used is fluorescent antibody for IFN-γ (purchased from BD, USA). The results are achieved by use of flow cytometry. The expression level of IFN-γ in CTL induced by DC which are respectively infected by AAV/HPV-16 E7ₘ viruses and AAV/HPV-16 E7 virus is shown in FIG. 10 (rAAV carrying SV40 early promoter as the representative for AAV/HPV-16 E7ₘ₉₁ viruses, rAAV carrying AAV p5 promoter as the representative for AAV/HPV-16 E7ₘ₉₄ viruses, rAAV carrying β-actin promoter as the representative for AAV/HPV-16 E7ₘ₅₈ viruses, and rAAV carrying CMV promoter as the representative for AAV/HPV-16 E7ₘₘ₃ viruses). The flow cytometry detection results of expression level of IFN-γ in CTL induced by DC which are respectively infected by AAV/HPV-16 E7ₘ viruses and AAV/HPV-16 E7 virus show that IFN-γ can be expressed in high level in CTL induced by DC infected by rAAV, which indicates that the CTL induced by DC which are infected by AAV/HPV-16 E7ₘ viruses or AAV/HPV-16 E7 virus of the present invention have strong ability to kill target cells and are more powerful.

The results of the above experiments B and C also demonstrate that AAV/HPV-16 E7ₘ is functionally equivalent to rAAV/HPV-16 E7 carrying the wild-type E7 antigen gene, and is capable of initiating an effective cellular immune response, i.e. not only can effectively stimulate function of DC, but also can lead to the production of CTL.

D. Tests of Cytotoxic-T-Lymphocytes (CTL) to Kill Tumor Cells

After mixedly culturing, the CTL induced by DC which are infected by AAV/HPV-16 E7ₘ viruses or AAV/HPV-16 E7 virus in step 1 are respectively mixed with cervical cancer cells, breast cancer cells, penile cancer cells, anal cancer cells and oral cancer cells at the ratio of 20:1 (lymphocytes:tumor cells). The cytotoxicity of CTL on tumor cells is detected by the traditional ⁵¹Cr release assay.

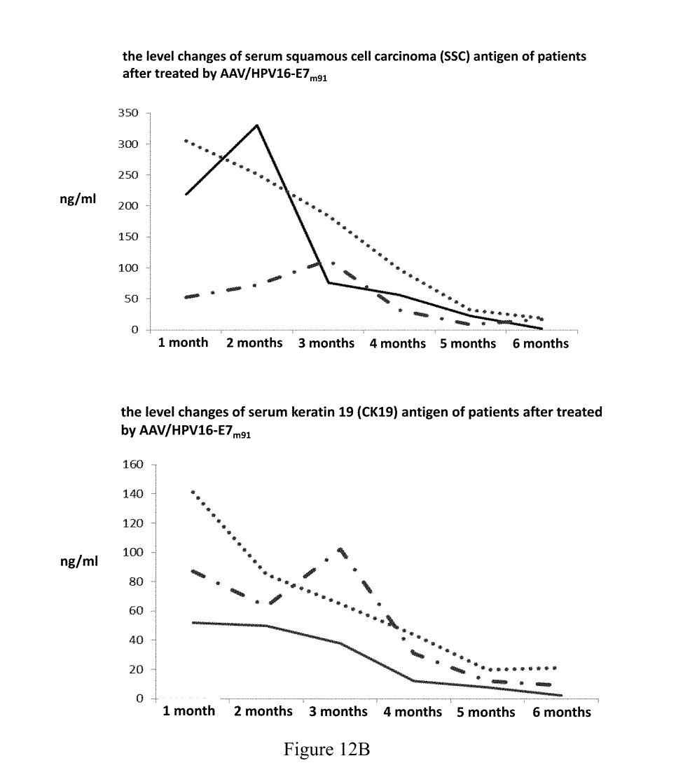

The ⁵¹Cr assay results of cytotoxicity of cytotoxic-T-lymphocytes (CTL) induced by DC which are infected by rAAV on HPV-16 E7 positive cells in vitro are shown in FIG. 11A-11G (taking AAV/HPV-16 E7ₘ carrying CMV promoter as an example, the ordinate indicates the kill rate). The ⁵¹Cr assay results of cytotoxicity of CTL induced by DC which are infected by AAV/HPV-16 E7ₘ carrying CMV promoter on HPV-16 E7 positive cells in vitro indicate that the CTL induced by DC which are infected by the rAAV of the present invention can effectively lyse (kill) HPV-16-E7 antigen positive tumor cells (target cells) at a killing rate of 40% to 70%.

The HPV-16-E7 negative cells of lung, breast, liver and kidney are used as controls; the same methods as mentioned above are used to detect the cytotoxicity specificity of CTL induced by DC which are infected by AAV/HPV-16 E7ₘ viruses or AAV/HPV-16 E7 virus. The results are also shown in FIG. 11A-11G (taking AAV/HPV-16 E7ₘ carrying CMV promoter as an example, the ordinate indicates the kill rate). The results indicate that CTL induced by DC which are infected by AAV/HPV-16 E7ₘ viruses or rAAV/HPV-16 E7 virus of the present invention have no cytotoxicity on cells of lung, breast, liver and kidney.

The tests prove that CTL induced by DC which are infected by AAV/HPV-16 E7ₘ viruses or rAAV/HPV-16 E7 virus of the present invention have antigenic specificity, i. e., not having cytotoxicity on cells which are antigen-negative.

The above-mentioned detection results show that CTLs induced by DC which are infected by rAAVs carrying mutant HPV-16 E7ₘ antigen gene (AAV/HPV-16 E7ₘ viruses) have strong killing (lysing) effect on HPV-16-E7 antigen positive cells and high specificity (i.e., targeted killing activity), while have no cytotoxicity on cells which are HPV-16-E7 antigen negative. There is no significant difference in cytotoxicity between CTLs induced by wild-type HPV-16 E7 antigen and mutant HPV-16 E7ₘ antigen. In addition, the CTLs of the present invention have no oncogenicity and can be used to produce antitumor drugs.

### Example 8: Clinical Tests of Treatment for HPV-16-E7 Antigen Positive Tumor

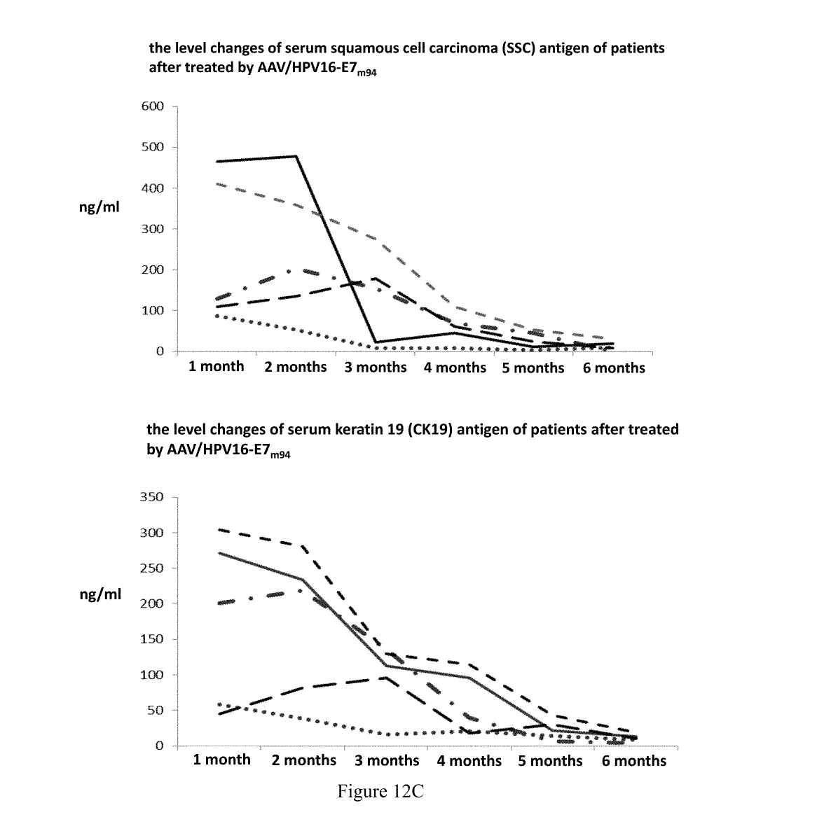

1. The rAAV-DC technology is used so that 5 cases of cervical cancer patients are reinfused with CTL induced by DC which are infected by AAV/HPV-16 E7ₘ₅₈ virus of the present invention. All patients have been confirmed to be HPV-16 E7 positive in their cervical cancer tissue. The infusion volume is 2×10⁸-5×10⁸. A treatment course is usually 3 months, 2 times per month, which can be reduced to 1-2 times per month, and further reduced to one treatment every 1-3 months with the medical improvements. The results of treatment are summarized in Table 1 (B: reduction or disappearance of serum tumor markers; Q: improvement in life quality of patients, such as relief or loss of pain, increased appetite, etc; C: significant reduction or disappearance of cancer lesions or metastatic lesions through CT or PET-CT). Adverse reactions: 1 case of mild flu-like response within a short time after treatment is observed, but it is tolerable to the subject, and the symptoms disappear in short time; no serious adverse reactions or toxic reactions are observed. The level changes of serum squamous cell carcinoma antigen (SCC) and serum keratin 19 antigen (CK19) of 5 patients with cervical cancer before and after treatment with CTL which are obtained by induction of the DC infected by rAAV/HPV-16 E7ₘ₅₈ virus are shown in FIG. 12A. After treatment, the serum levels of serum keratin 19 antigen (CK19, cyfra21-1) and serum squamous cell carcinoma antigen (SCC) decrease significantly in 3 patients (CK19 level, normal value<3.3 ng/mL, SCC level, normal value<5.0 ng/mL), or even return to normal level.

At the time of filing the present invention, 5 patients all survive. The results of clinical tests further demonstrate that the CTL induced by rAAV-infected DC of the present invention can exert certain therapeutic effect in the patient and can effectively inhibit the growth of HPV-16 E7 positive malignant tumor cells or kill tumor cells. The CTL induced by rAAV-infected DC of the present invention have high safety and can be used for the preparation of anti-tumor drugs.

TABLE 1
Statistic results of therapeutic effect of rAAV/HPV-16-
E7ₘ₅₈-DC therapy in 5 patients with cervical cancer

| | Clinical | rAAV-DC reatment | Survival time after | Therapeutic |
|---|---|---|---|---|
| No. | Phases | course (month) | treatment (month) | effect |
| 1 | III | 9 | 17 | B, Q, C |
| 2 | III | 11 | 26 | B, Q, C |
| 3 | III | 8 | 13 | Q |
| 4 | IV | 8 | 12 | B, Q |
| 5 | IV | 6 | 14 | Q, |
| Sum | III-IV | 42 | 82 | |

2. The rAAV-DC technology is used so that 3 cases of advanced cervical cancer patients are reinfused with CTL induced by DC which are infected by AAV/HPV-16 E7ₘ₉₁ virus of the present invention. All patients have been confirmed to be HPV-16 E7 positive in their cervical cancer tissue. The infusion volume is 2×10⁸-5×10⁸. A treatment course is usually 3 months, 2 times per month. The results of treatment are summarized in Table 2 (B: reduction or disappearance of serum tumor markers; Q: improvement in life quality, such as relief or loss of pain, increased appetite, etc; C: significant reduction or disappearance of cancer lesions or metastatic lesions through CT or PET-CT). Adverse reactions: no serious adverse reactions or toxic reactions are observed.

The level changes of serum keratin 19 antigen (CK19) and serum squamous cell carcinoma antigen (SCC) of 3 advanced cervical cancer patients before and after treated by CTL which are obtained by the induction of the DC infected by AAV/HPV-16 E7ₘ₉₁ virus are shown in FIG. 12B. After treatment, the serum levels of serum keratin 19 antigen (CK19, cyfra21-1) and serum squamous cell carcinoma antigen (SCC) decrease significantly in 3 patients (CK19 level, normal value<3.3 ng/mL, SCC level, normal value<5.0 ng/mL), or even return to normal level. At the time of filing the present invention, 3 patients all survive. The results of clinical trials further demonstrate that the CTL induced by rAAV-infected DC of the present invention can exert a certain therapeutic effect in the patient and can effectively inhibit the growth of HPV-16 E7 positive malignant tumor cells or kill tumor cells. The CTL induced by rAAV-infected DC of the present invention have high safety and can be used for the preparation of anti-tumor drugs.

TABLE 2
Statistic results of therapeutic effect of rAAV/HPV-16-E7ₘ₉₁-dendritic-
cell (rAAV-DC) therapy in 3 patients with cervical cancer

| | Clinical | rAAV-DC reatment | Survival time after | Therapeutic |
|---|---|---|---|---|
| No. | phases | course (month) | treatment (month) | effect |
| 1 | III | 6 | 11 | B, Q, C |
| 2 | III | 4 | 10 | B, Q |
| 3 | III | 5 | 15 | B, Q, C |
| Sum | III | 15 | 36 | |

3. The rAAV-DC technology is used so that 5 cases of cervical cancer patients are reinfused with CTL induced by DC which are infected by AAV/HPV-16 E7ₘ₉₄ virus of the present invention in Example 4. All patients have been confirmed to be HPV-16 E7 positive in cervical cancer tissue. The infusion volume is 2×10⁸-5×10⁸. The treatment course is usually 3 months, 2 times per month. The results of treatment are summarized in Table 3 (B: reduction or disappearance of serum tumor markers; Q: improvement in life quality of patients, such as relief or loss of pain, increased appetite, etc; C: significant reduction or disappearance of cancer lesions or metastatic lesions through CT or PET-CT). Adverse reactions: 1 case of mild flu-like response within a short time after treatment is observed, but it is tolerable to the subject, and the symptoms disappear in short time; no serious adverse reactions or toxic reactions are observed.

The level changes of serum keratin 19 antigen (CK19) and serum squamous cell carcinoma antigen (SCC) of 5 cervical cancer patients before and after treated by CTL which are obtained by induction of the DC infected by AAV/HPV-16 E7ₘ₉₄ virus are shown in FIG. 12C. After treatment, the serum levels of serum keratin 19 antigen (CK19, cyfra21-1) and serum squamous cell carcinoma antigen (SCC) decrease significantly in 3 patients (CK19 level, normal value<3.3 ng/mL, SCC level, normal value<5.0 ng/mL), or even return to normal level. The results of clinical trials further demonstrate that the CTL induced by rAAV-infected DC of the present invention can exert a certain therapeutic effect in the patient and can effectively inhibit the growth of HPV-16 E7 positive malignant tumor cells or kill tumor cells. The CTL induced by rAAV-infected DC of the present invention have high safety and can be used for the preparation of anti-tumor drugs.

TABLE 3
Statistic results of therapeutic effect of rAAV/HPV-16-
E7ₘ₉₄-dendritic-cell therapy in 5 patients with cervical cancer

| | Clinical | rAAV-DC treatment | Survival time after | Therapeutic |
|---|---|---|---|---|
| No. | phases | course (month) | treatment (month) | effect |
| 1 | II | 3 | 12 | B, Q, C |
| 2 | III | 6 | 8 | B, Q, C |
| 3 | III | 6 | 9 | B, Q |
| 4 | III | 6 | 14 | Q, C |
| 5 | IV | 6 | 10 | B, Q |
| Sum | III-IV | 27 | 53 | |

4. The rAAV-DC technology is used so that 5 cases of cervical cancer patients, 2 cases of anal cancer patients and 4 cases of penile cancer patients are reinfused with CTL induced by DC which are infected by AAV/HPV-16 E7 virus or AAV/HPV-16 E7ₘₘ viruses of the present invention. All patients have been confirmed to be HPV-16 E7 positive in their cancer tissue. The infusion volume is 2×10⁸-5×10⁸. A treatment course is usually 6 months, 2 times per month which can be reduced to 1-2 times per month, and further reduced to one treatment every 1-3 months with the medical improvements.

The results of treatment are summarized in Table 4-6 (B: reduction or disappearance of serum tumor markers; Q: improvement in life quality of patients, such as relief or loss of pain, increased appetite, etc; C: significant reduction or disappearance of cancer lesions or metastatic lesions through CT or PET-CT). Adverse reactions: no serious adverse reactions or toxic reactions are observed.

The level changes of serum CK19 (cyfra21-1, normal value<3.3 ng/mL) and SCC (normal value<5.0 ng/mL) of 5 cervical cancer patients before and after treated by CTL which are obtained by the induction of the DC infected by AAV/HPV-16 E7ₘₘ₃ virus are shown in FIG. 12D (the ordinate indicates the concentration, ng/mL). After treatment, the levels of serum keratin 19 antigen (CK19, cyfra21-1, normal value<3.3 ng/mL) and serum squamous cell carcinoma antigen (SCC, normal value<5.0 ng/mL) decrease significantly, or even return to normal level.

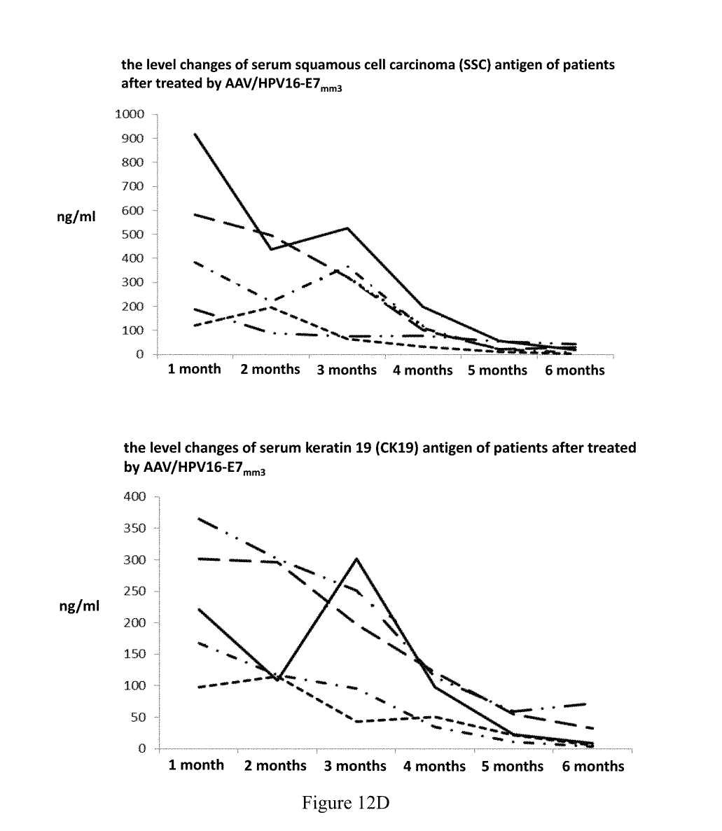

The level changes of serum CK19 (cyfra21-1, normal value<3.3 ng/mL) of 2 anal cancer patients before and after treated by CTL which are obtained by the induction of the DC infected by AAV/HPV-16 E7ₘₘ₃ virus are shown in FIG. 13 (the ordinate indicates the concentration, ng/mL). After treatment by CTL which are obtained by the induction of the DC infected by AAV/HPV-16 E7ₘₘ₃ virus, the levels of serum keratin 19 antigen (CK19, cyfra21-1, normal value<3.3 ng/mL) in anal cancer patients all decrease, indicating that the treatment is effective.

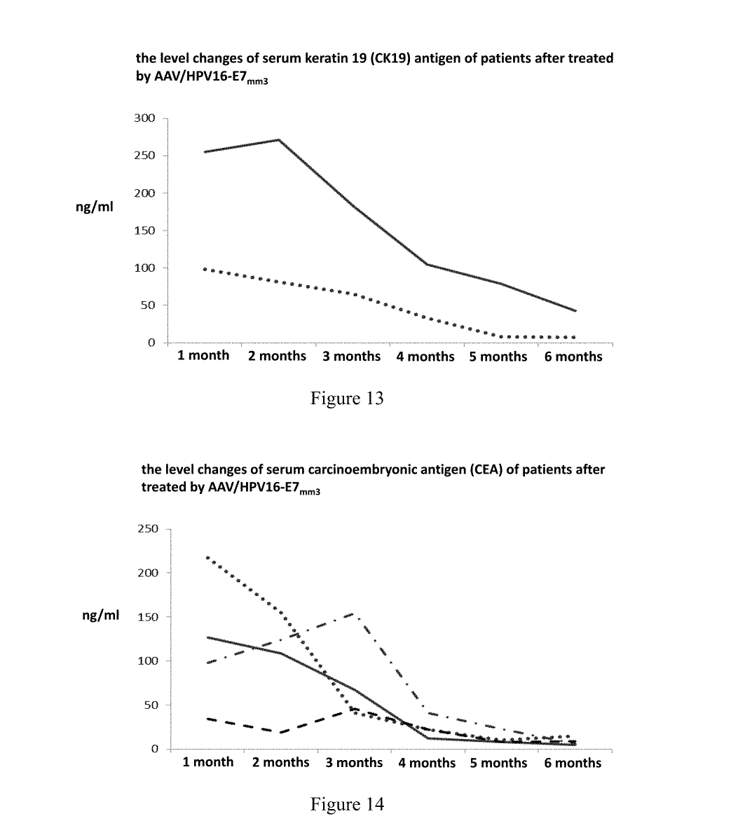

The level changes of serum carcinoembryonic antigen (CEA, normal value<5.0 ng/mL) in 4 penile cancer patients before and after treated by CTL which are obtained by the induction of the DC infected by AAV/HPV-16 E7ₘₘ₃ virus are shown in FIG. 14 (the ordinate indicates the concentration, ng/mL). After treatment by CTL which are obtained by the induction of the DC infected by AAV/HPV-16 E7ₘₘ₃ virus, the levels of serum carcinoembryonic antigen (CEA, normal value<5.0 ng/mL) in penile cancer patients all decrease, indicating that the treatment is effective.

The experiment results show that the serum tumor markers (tumor-associated antigen) all decrease significantly in patients, or even returned to normal. The results of clinical tests further demonstrate that the CTL induced by rAAV-infected DC (collectively called rAAV-DC) of the present invention can exert a certain therapeutic effect in the patient and can effectively inhibit the growth of HPV-16 E7 positive malignant tumor cells or kill tumor cells. The CTL induced by rAAV-DC of the present invention have high safety and can be used for the preparation of anti-HPV-16 infection and anti-tumor drugs for HPV-16-E7-positive tumors.

TABLE 4
Statistic results of therapeutic effect of rAAV/HPV-16-E7ₘₘ₃-dendritic-
cell (rAAV-DC) therapy in patients with cervical cancer

| | Clinical | rAAV-DC reatment | Survival time after | Therapeutic |
|---|---|---|---|---|
| No. | phases | course (month) | treatment (month) | effect |
| 1 | III | 7 | 10 | B, Q, C |
| 2 | III | 6 | 9 | B, Q |
| 3 | III | 9 | 9 | B, Q |
| 4 | IV | 9 | 12 | Q, C |
| 5 | IV | 12 | 15 | B, Q |
| Sum | III-IV | 43 | 55 | |

TABLE 5
Statistic results of therapeutic effect of rAAV/HPV-16-E7ₘₘ₃-dendritic-
cell (rAAV-DC) therapy in patients with anal cancer

| | Clinical | rAAV-DC reatment | Survival time after | Therapeutic |
|---|---|---|---|---|
| No. | phases | course (month) | treatment (month) | effect |
| 1 | III | 6 | 12 | B, Q, C |
| 2 | IV | 8 | 10 | B, Q |
| Sum | III-IV | 14 | 22 | |

TABLE 6
Statistic results of therapeutic effect of rAAV/HPV-16-E7ₘₘ₃-dendritic-
cell (rAAV-DC) therapy in patients with penile cancer

| | Clinical | rAAV-DC reatment | Survival time after | Therapeutic |
|---|---|---|---|---|
| No. | phases | course (month) | treatment (month) | effect |
| 1 | II | 9 | 12 | B, Q, C |
| 2 | III | 6 | 10 | B, Q, C |
| 3 | IV | 12 | 15 | B, Q, C |
| 4 | IV | 3 | 6 | B, Q |
| Sum | III-IV | 47 | 55 | |

### INDUSTRIAL APPLICABILITY

Experiments have shown that dendritic cells infected by rAAV carrying HPV-16 E7ₘ of the present invention and the induced cytotoxic-T-lymphocytes can effectively inhibit the growth of HPV16-infected cells and related malignant tumor cells thereof or can kill the tumor cells. Thus, the rAAV carrying HPV-16 E7ₘ of the present invention or the products associated with the rAAV carrying HPV-16 E7ₘ of the present invention can be used to prepare anti-HPV-16 infection drugs and associated anti-tumor drugs. the rAAV carrying HPV-16 E7ₘ of the present invention or the products associated with the rAAV carrying HPV-16 E7ₘ of the present invention are of great importance for clinical treatment and application.

1. Recombinant adeno-associated virus vector carrying mutant HPV-16 E7 antigen gene, obtained by inserting a mutant HPV-16 E7 antigen gene, named as HPV-16 E7ₘ, into a original vector; wherein the original vector is an AAV vector from which the adeno-associated virus structural genes Rep and Cap in the adeno-associated virus (AAV) vector have been eliminated and by which any one of p5 promoter, cytomegalovirus promoter, SV40 virus promoter and β-actin promoter of AAV is carried; the recombinant adeno-associated virus vector is a new rAAV vector carrying HPV-16 E7ₘ antigen gene and is named as AAV/HPV-16 E7ₘ.

2. The recombinant adeno-associated virus vector according to claim 1, wherein, the said mutant HPV-16 E7ₘ antigen gene is a coding gene obtained correspondingly by replacing one, two or three of the cysteine (C) at position 58, 91 and 94 of the HPV-16 E7 antigen protein with glycine (G).

3. The recombinant adeno-associated virus vector according to claim 2, wherein, the said mutant HPV-16 E7ₘ antigen gene is obtained by replacing one, two or three of the thymine (T) at position nt175, nt271 and nt280 of the open reading frames of HPV-16 E7 antigen gene with guanine (G).

4. The recombinant adeno-associated virus vector according to claim 1, wherein, the said mutant HPV-16 E7ₘ antigen gene is one of the follows:
A mutant HPV-16 E7 gene with one mutation site of nt175 (58aa), named as HPV-16 E7ₘ₅₈, and the nucleotide sequence thereof is shown in SEQ ID NO: 2 in the Sequence Listing or FIG. 3A;
A mutant HPV-16 E7 gene with one mutation site of nt271 (91aa), named as HPV-16 E7ₘ₉₁, and the nucleotide sequence thereof is shown in SEQ ID NO: 3 in the Sequence Listing or FIG. 3B;
A mutant HPV-16 E7 gene carrying one mutation site of nt280 (94aa), named as HPV-16 E7ₘ₉₄, and the nucleotide sequence thereof is shown in SEQ ID NO: 4 in the Sequence Listing or FIG. 3C;
A multiple-site-mutant HPV-16 E7 gene with two mutation sites of nt175 (58aa) and nt271 (91aa), named as HPV-16 E7ₘₘ₂₁, and the nucleotide sequence thereof is shown in SEQ ID NO: 5 in the Sequence Listing or FIG. 3D;
A multiple-site-mutant HPV-16 E7 gene with two mutation sites of nt175 (58aa) and nt280 (94aa), named as HPV-16 E7ₘₘ₂₂, and the nucleotide sequence thereof is shown in SEQ ID NO: 6 in the Sequence Listing or FIG. 3E;
A multiple-site-mutant HPV-16 E7 gene with two mutation sites of nt271 (91aa) and nt280 (94aa), named as HPV-16 E7ₘₘ₂₃, and the nucleotide sequence thereof is shown in SEQ ID NO: 7 in the Sequence Listing or FIG. 3F;
A multiple-site-mutant HPV-16 E7 gene with three mutation sites of nt175 (58aa), nt271 (91aa) and nt280 (94aa), named as HPV-16 E7ₘₘ₃, and the nucleotide sequence thereof is shown in SEQ ID NO: 8 in the Sequence Listing or FIG. 3G.

5. The recombinant adeno-associated virus vector according to claim 1, wherein, the said adeno-associated virus vector is an adeno-associated virus vector from which the adeno-associated virus (AAV) structural genes Rep and Cap have been eliminated, and the promoter carried by the adeno-associated virus vector is any one of p5 promoter, cytomegalovirus promoter, human β-actin promoter and SV40 virus early promoter has been carried.

6. A method for constructing the said recombinant adeno-associated virus vector according to claim 1, comprising the steps of:
1) replacing one, two or three of cysteine (C) at position 58, 91 and 94 of the HPV-16 E7 antigen with glycine (G), i.e., replacing one, two or three of thymine (T) at position nt175, nt271 and nt280 of the open reading frames of HPV-16 E7 gene with guanine (G), to obtain a mutant HPV-16 E7 antigen gene with one, two or three mutation sites, uniformly named as HPV-16 E7ₘ; and
2) respectively inserting the mutant HPV-16 E7ₘ antigen gene or wild-type HPV-16 E7 antigen gene to an adeno-associated virus vector from which the adeno-associated virus structural genes Rep and Cap have been eliminated to obtain a recombinant adeno-associated virus vector carrying the mutant HPV-16 E7ₘ antigen gene, named as AAV/HPV-16 E7ₘ; or a recombinant adeno-associated virus vector carrying HPV-16 E7 antigen gene, named as AAV/HPV-16 E7, respectively; the promoter carried by the adeno-associated virus vector is any one of AAV p5 promoter, cytomegalovirus promoter, human β-actin promoter or SV40 virus early promoter.

7. The method according to claim 6, wherein, the specific procedure of step 1) is as any one of the following:
1). Cysteine (C) at position 58 of HPV-16 E7 antigen protein is replaced with glycine (G), the detailed procedure is to replace the thymine (T) at position nt175 of the open reading frames of HPV-16 E7 antigen gene with guanine (G), i. e., to replace the “tgc” encoding cysteine, at position nt175-177, with “ggc” encoding glycine, to obtain a HPV-16 E7 antigen gene with one mutation site, and named as HPV-16 E7ₘ₅₈; the obtained recombinant adeno-associated virus vector carrying the mutant HPV-16 E7ₘ₅₈ antigen gene is named as AAV/HPV-16 E7ₘ₅₈;
2). Cysteine (C) at position 91 of HPV-16 E7 antigen protein is replaced with glycine (G); the detailed procedure is to replace the thymine (T) at position nt271 of the open reading frames of HPV-16 E7 antigen gene with guanine (G), i. e., to replace the “tgc” encoding cysteine at position nt271-273 with “ggc” encoding glycine, to obtain a HPV-16 E7 antigen gene with one mutation site, and named as HPV-16 E7ₘ₉₁; the obtained recombinant adeno-associated virus vector carrying the mutant HPV-16 E7ₘ₉₁ antigen gene is named as AAV/HPV-16 E7ₘ₉₁;
3). Cysteine (C) at position 94 of HPV-16 E7 antigen protein is replaced with glycine (G); the detailed procedure is to replace the thymine (T) at position nt280 of the open reading frames of HPV-16 E7 antigen gene with guanine (G), i. e., to replace the “tgt” encoding cysteine at position nt271-273 with “ggt” encoding glycine, to obtain a HPV-16 E7 antigen gene with one mutation site, named as HPV-16 E7ₘ₉₄; the obtained recombinant adeno-associated virus vector carrying the mutant HPV-16 E7ₘ₉₄ antigen gene is named as AAV/HPV-16 E7ₘ₉₄;
4). Cysteines (C) at position 58 and 91 of HPV-16 E7 antigen protein are replaced with glycines (G); the detailed procedure is to replace the thymines (T) at position nt175 and nt271 of the open reading frames of HPV-16 E7 antigen gene with guanines (G), i. e., to replace the “tgc” encoding cysteine at position nt175-177 and nt271-273 with “ggc” encoding glycine, to obtain a HPV-16 E7 antigen gene with two mutation sites, and named as HPV-16 E7ₘₘ₂₁; the obtained recombinant adeno-associated virus vector carrying the double-site-mutant HPV-16 E7ₘₘ₂₁ antigen gene is named as AAV/HPV-16 E7ₘₘ₂₁;
5). Cysteines (C) at position 58 and 94 of HPV-16 E7 antigen protein are replaced with glycines (G); the detailed procedure is to replace the thymines (T) at position nt175 and nt280 of the open reading frames of HPV-16 E7 antigen gene with guanines (G), i. e., to replace the “tgc” and “tgt” encoding cysteine respectively at position nt175-177 and nt280-282 with “ggc” and “ggt” encoding glycine respectively, to obtain a HPV-16 E7 antigen gene with two mutation sites, and named as HPV-16 E7ₘₘ₂₂; the obtained recombinant adeno-associated virus vector carrying the double-site-mutant HPV-16 E7ₘₘ₂₂ antigen gene is named as AAV/HPV-16 E7ₘₘ₂₂;
6). Cysteines (C) at position 91 and 94 of HPV-16 E7 antigen protein are replaced with glycines (G); the detailed procedure is to replace the thymines (T) at position nt271 and nt280 of the open reading frames of HPV-16 E7 antigen gene with guanines (G), i. e., to replace the “tgc” and “tgt” encoding cysteine respectively at position nt271-273 and nt280-282, with “ggc” and “ggt” encoding glycine respectively, to obtain a HPV-16 E7 antigen gene with two mutation sites, and named as HPV-16 E7ₘₘ₂₃; the obtained recombinant adeno-associated virus vector carrying the double-site-mutant HPV-16 E7ₘₘ₂₃ antigen gene is named as AAV/HPV-16 E7ₘₘ₂₃;
7). Cysteines (C) at position 58, 91 and 94 of HPV-16 E7 antigen protein are replaced with Glycines (G); the detailed procedure is to replace the thymines (T) at position nt175, nt271 and nt280 of the open reading frames of HPV-16 E7 antigen gene with guanines (G), i. e., to replace the “tgc”, “tgc” and “tgt” encoding cysteine, respectively at position nt175-177, nt271-273 and nt280-282, with “ggc”, “ggc” and “ggt” encoding glycine respectively, to obtain a HPV-16 E7 antigen gene with three mutation sites, and named as HPV-16 E7ₘₘ₃; the obtained recombinant adeno-associated virus vector carrying the triple-site-mutant HPV-16 E7ₘₘ₃ antigen gene is named as AAV/HPV-16 E7ₘₘ₃.

8. Products associated with the recombinant adeno-associated virus vector of claim 1, the products include recombinant adeno-associated virus plasmids, recombinant adeno-associated virus particles and cell lines infected or transfected by the recombinant adeno-associated virus vector; the cell lines include monocytes (Mo) and dendritic cells (DC).

9. Methods for preparing the products of claim 8, which are respectively as follows:
Preparation of recombinant adeno-associated virus plasmids: DNA of the recombinant adeno-associated virus vector, AAV/HPV-16 E7 or AAV/HPV-16 E7ₘ, are respectively introduced into genetically engineered Escherichia coli DH5a competent cells; and resistance screening is performed by LB agar plates with 100 g/mL ampicillin and the white colonies are picked, then the plasmids of the picked colonies are extracted and purified to obtain the AAV/HPV-16 E7 plasmids and AAV/HPV-16 E7ₘ plasmids;
Preparation of recombinant adeno-associated virus: the pHelper plasmid and the recombinant adeno-associated virus plasmids AAV/HPV-16 E7 plasmids or AAV/HPV-16 E7ₘ plasmids are used to co-transfect the AAV-HEK293 cells to obtain the recombinant adeno-associated virus, named as AAV/HPV-16 E7 virus and AAV/HPV-16 E7ₘ virus respectively;
Preparation of the cell lines infected or transfected by the recombinant adeno-associated virus: the recombinant adeno-associated virus, AAV/HPV-16 E7 virus or AAV/HPV-16 E7ₘ virus, are used to infect or transfect monocytes (Mo), dendritic cells (DC) or lymphocytes, respectively or in turn, to obtain the cell lines.

10. A cellular immunotherapy drug for anti-HPV-16 infection and a malignant tumor caused by HPV-16 infection, the active ingredient of the drug is the recombinant adeno-associated virus vector of claim 1.

11. The drug according to claim 10 wherein, the malignant tumor caused by HPV-16 infection is selected from HPV-16-E7-antigen-positive cervical papilloma lesions, cervical cancer, male genital Bowen's disease, giant condyloma accuminata, penile cancer, anal cancer, rectum cancer, oral cancer, tonsillar cancer, mammary cancer, etc.

12. A method for killing HPV-16-infected cells and HPV-16-E7-positive tumor cells, comprising
1) obtaining treated cells respectively by infecting or transfecting the monocytes isolated from a patient using the recombinant adeno-associated virus vector of claim 1 or treating the monocytes isolated from a patient; and
2) reinfusing the dendritic cells (DC) induced by the treated monocytes (Mo) of 1) into the patient to activate cytotoxic-T-lymphocytes (CTLs), which kill HPV-16-infected cells and HPV-16-E7-positive tumor cells; or mixedly culturing the non-treated T-lymphocytes and the treated Mo-DC to obtain HPV-16-E7-antigen-specific CTLs, which are then reinfused into the patient to kill the HPV-16-infected cells and HPV-16-E7-positive tumor cells; or reinfusing the treated T-lymphocytes and the treated Mo-DC into the patient to kill the HPV-16-infected cells and HPV-16-E7-positive tumor cells.


SEQUENCE LISTING (8 sequences)

SEQ ID NO: 1 - DNA, 297 bp, Human papillomavirus
atgcatggagatacacctacattgcatgaatatatgttagatttgcaaccagagacaactgatctctactgttatgagcaattaagtgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttcccagaaaccataa

SEQ ID NO: 2 - DNA, 297 bp, Human papillomavirus
atgcatggagatacacctacattgcatgaatatatgttagatttgcaaccagagacaactgatctctactgttatgagcaattaagtgacagctcagaggaggaggatgaaatagatggtccagctggacaagcagaaccggacagagcccattacaatattgtaaccttttgtggcaagtgtgactctacgcttcggttgtgcgtacaaagcacacacgtagacattcgtactttggaagacctgttaatgggcacactaggaattgtgtgccccatctgttcccagaaaccataa

SEQ ID NO: 3 - DNA, 297 bp, Human papillomavirus
atgcatggagatacacctacattgcatgaatatatgttagatttgcaaccagagacaactgatctctactgttatgagcaattaagtgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgggccccatctgttcccagaaaccataa

SEQ ID NO: 4 - DNA, 297 bp, Human papillomavirus
atgcatggagatacacctacattgcatgaatatatgttagatttgcaaccagagacaactgatctctactgttatgagcaattaagtgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatcggttcccagaaaccataa

SEQ ID NO: 5 - DNA, 297 bp, Human papillomavirus
atgcatggagatacacctacattgcatgaatatatgttagatttgcaaccagagacaactgatctctactgttatgagcaattaagtgacagctcagaggaggaggatgaaatagatggtccagctggacaagcagaaccggacagagcccattacaatattgtaaccttttgtggcaagtgtgactctacgcttcggttgtgcgtacaaagcacacacgtagacattcgtactttggaagacctgttaatgggcacactaggaattgtgggccccatctgttcccagaaaccataa

SEQ ID NO: 6 - DNA, 297 bp, Human papillomavirus
atgcatggagatacacctacattgcatgaatatatgttagatttgcaaccagagacaactgatctctactgttatgagcaattaagtgacagctcagaggaggaggatgaaatagatggtccagctggacaagcagaaccggacagagcccattacaatattgtaaccttttgtggcaagtgtgactctacgcttcggttgtgcgtacaaagcacacacgtagacattcgtactttggaagacctgttaatgggcacactaggaattgtgtgccccatcggttcccagaaaccataa

SEQ ID NO: 7 - DNA, 297 bp, Human papillomavirus
atgcatggagatacacctacattgcatgaatatatgttagatttgcaaccagagacaactgatctctactgttatgagcaattaagtgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgggccccatcggttcccagaaaccataa

SEQ ID NO: 8 - DNA, 297 bp, Human papillomavirus
atgcatggagatacacctacattgcatgaatatatgttagatttgcaaccagagacaactgatctctactgttatgagcaattaagtgacagctcagaggaggaggatgaaatagatggtccagctggacaagcagaaccggacagagcccattacaatattgtaaccttttgtggcaagtgtgactctacgcttcggttgtgcgtacaaagcacacacgtagacattcgtactttggaagacctgttaatgggcacactaggaattgtgggccccatcggttcccagaaaccataa